(19) United States
(12) Patent Application Publication
(10) Pub. No.: US 2002/0102232 A1
(43) Pub. Date: Aug. 1, 2002

(54) Compositions and methods for induction of active autoimmunity

(76) Inventors: Tse W. Chang, Taipei (TW); Jim J.C. Sheu, Hua-Lien (TW); Janice S.W. Huang, Hsinchu (TW); Stanley C.S. Wu, Taichung (TW); Leslie Y.Y. Chen, Miaoli (TW)

Correspondence Address: LAHIVE & COCKFIELD, 28 STATE STREET, BOSTON, MA, 02109, US

(21) Appl. No.: 09/852,976

(22) Filed: May 10, 2001

Related U.S. Application Data
(60) Provisional application No. 60/204,191, filed on May 11, 2000.

(30) Foreign Application Priority Data
May 11, 2000 (US) PCT/US00/12997

(51) Int. Cl. A61K 39/395; A61K 38/19; C07H 21/04; C12P 21/02; C12N 5/06

(52) U.S. Cl. 424/85.1; 424/178.1; 424/185.1; 435/69.5; 435/325; 435/320.1; 536/23.5

(57) ABSTRACT

A recombinant first antigen, coupled with a foreign protein (such as immunoglobulin Fc from different species), can be used as a vaccine to induce active auto-immunity specifically through a T cell-dependent antibody response. The induced autoantibodies can recognize self-antigen in vivo and trigger immune responses to reduce or eliminate a target autologous antigen. Since there is evidence that the pathogenesis of some diseases, such as cancer, allergy, arthritis, atherosclerosis, graft rejection, or other inflammatory diseases, are caused by increased levels of certain autologous proteins, the instant compositions and methods provide a method of inducing autoantibodies to down-regulate the levels of a target autologous antigen or cells expressing the antigen to ameliorate diseases or disorders.

### RELATED APPLICATIONS

This application claims priority to U.S. provisional application Serial No. 60/204,191 filed May 11, 2000, and to International Application, PCT/US00/12997 filed on May 11, 2000, the contents of which are entirely incorporated by reference.

### BACKGROUND OF THE INVENTION

Expression of certain plasma proteins, cytokines, receptors, or cell surface molecules has been correlated with increased severity of some diseases. Control of the level of expression of these autologous molecules could be used to control progression of such diseases.

Monoclonal antibodies have been considered a good class of natural drugs for the application in therapy of human diseases. Antibody drugs can be used to neutralize toxins produced by microorganisms, opsonize viruses and bacteria to abolish their ability to bind to host cells, kill tumor cells, etc. In most cases, the therapeutic targets recognized by antibody drugs are expressed on the surface of target cells, such as specific tumor markers. Other examples include: OKT3, a potent immunosupressive agent with marked efficacy in the treatment of tissue allograft rejection (Cosimi, et al., 1990, Arch. Surg. 125:781-785, and Hammond, et al., 1990, J. Heart Transplant. 9:236-238); Rituxan, a chimeric monoclonal antibody that binds specifically to the CD20 antigen, (Coiffier, et al., 1998, Blood, 92:1927-1932, and U.S. Pat. No. 5,736,137); and anti-IgE, a monoclonal antibody drug used to treat patients with moderate to severe forms of asthma without the side effects of current treatments, such as steroids and muscle relaxants (King, R. T., 1999, Wall Street J. Interactive Ed., December 20) have been developed.

However, antibodies developed in species other than man elicit potent immune responses in human subjects and limit the efficacy of such therapies over the long term. See Willians, R. O. et al, Immunology 84:433-439 (1995); Elliott, M. J. et al., Lancet 344:1125-1127 (1994). Many antibody drugs employ chimerized or humanized forms of antibodies that are designed to improve their therapeutic effects by reducing their immunogenicity (Vaughan, et al., 1998, Nat. Biotech. 16:535-539). However, monoclonal antibodies and engineered recombinant proteins for clinical applications are impractical to manufacture in large amounts.

In addition, although the therapeutic effect of these antibodies is promising, patients must be treated with relatively high dosages of antibodies (about 300-500 mg/m²) and must be repeatedly injected with the antibodies (Maloney, et al., 1997, J. Clin. Oncol. 15:3266-3274, and Maloney, et al., 1997, Blood. 90:2188-2195). Continuous weekly infusions are necessary because antibodies are quickly metabolized.

Selective targeting of autologous antigens could be accomplished by inducing autoimmunity to selected antigens in a subject. Various prior art approaches have been attempted to induce autoimmunity. Some autoimmunogenic chemicals have been identified, such as mercury, silver, diphenyhydation (DPH), to induce autoimmunity and cause autoimmune disease-like syndrome in mice. See Gran, B. et al., Ann Neurol. 45:559-67 (1999); Johansson, U. et al., Int. Arch. Allergy Immunol. 113:432-43 (1997); Albers, R. et al., Immunol. 89:468-73 (1996). Some transgenic mice have also been prepared for research into the mechanism of inducing autoimmunity. See Feuerstein, N. et al., J Immunol.163:5287-97 (1999); Iwakura, Y. et al., J Immunol. 155:1588-98 (1995); Peng, S. L. et al., J. Immunol. 157:5225-30 (1996).

Viral infection (protein mimicry), shaping of T cell determinants, X-irradiation, and idiotypic induction have also been demonstrated as possible mechanisms for induction of autoimmunity. See Morse, S. S. et al., J. Immunol. 162:5309-16 (1999); Manns, M. P. et al., J. Viral. Hepat. 4:42-7 (1997); Moudgil, K. D. et al., Immunol. Lett. 68:251-6 (1999); Reynolds, P. et al., J. Exp. Med. 184:1857-70 (1996); Beijleveld, L. J. et al., Transplantation 59:1601-5 (1995); Sakaguchi, N. et al., J. Immunol. 152:2586-95 (1994); Krause, I. Et al., J. Biol. Regul. Homeost. Agents 12:49-52 (1998); Shoenfeld, Y. et al., FASEB J. 8:1296-301 (1994).

However, the prior art approaches do not lead to the induction of specific autoimmune responses against selected target antigens. Clearly, the development of novel compositions and methods for controlling the expression and/or activity of autologous antigens would be of great benefit.

### SUMMARY

The instant compositions and methods induce an active immune response against an autologous target antigen. The induction of a specific, active immune response against autologous antigens provides advantages over the prior art methods and can result in the reduction or elimination of those antigens or in the reduction or elimination of cells which bear them. The ability to induce an active, specific immune response eliminates the need for passive infusions of large quantities of antibodies. In addition, the instant compositions do not need to be administered frequently and require lower dosages per administration. Therefore, in addition to their other advantages, the instant compositions are more cost-effective and are easier to produce.

In one aspect, the invention provides immunogenic compositions, comprising a first polypeptide coupled to a second polypeptide, wherein the second polypeptide is heterologous to a subject, the composition being capable of eliciting an immune response against an autologous antigen in the subject.

In another aspect, the invention provides immunogenic compositions, comprising a first polypeptide, wherein the first polypeptide is sufficiently homologous to an autologous polypeptide in a subject, coupled to a second polypeptide, wherein the second polypeptide is heterologous to the subject, the composition being capable of eliciting an immune response against an autologous antigen in the subject.

In yet another aspect, the invention provides immunogenic compositions, comprising a first polypeptide, which is autologous to a subject, coupled to a second polypeptide, which is heterologous to the subject, the composition being capable of eliciting an immune response against an autologous antigen in the subject.

In one embodiment, the subject is a human.

In another embodiment, the autologous antigen is a cell-associated antigen. In yet another embodiment, the autologous antigen is a cell surface receptor.

In still another embodiment, the autologous antigen is a soluble antigen. In yet another embodiment, the autologous antigen is a cytokine or a hormone.

In another embodiment, the autologous antigen is selected from the group consisting of: CD64, sL-selectin, elastase, sCD16, CD46, TNF-α, sTNF-R75, sTNF-R55, TGF-β, CD40, CD154, lipoprotein (a), CD56, IL-10, IFN-γ, IL-2, IL-2R, CD45, IL-4, IgE, EGFR, TGF-β, CD54, sCD44 v5, and CD95.

In another embodiment, the autologous antigen is a tumor-associated antigen.

In still another embodiment, the autologous antigen is expressed by a B cell. In yet another embodiment, the autologous antigen is expressed specifically by B cells. In another embodiment, the autologous antigen is expressed specifically by activated B cells.

In one embodiment, the first polypeptide and the second polypeptide are expressed as a fusion protein. In another embodiment, the fusion protein is dimeric. In yet another embodiment, the first polypeptide and the second polypeptide are coupled via a chemical linkage.

In one embodiment, the first polypeptide comprises at least a portion of a molecule selected from the group consisting of: CD79α, CD79β, CD20, and Ig.

In one embodiment, the second polypeptide comprises at least one T helper cell epitope. In one embodiment, the second polypeptide comprises at least a portion of an Fc region of an immunoglobulin molecule.

In another aspect, the instant invention is directed to compositions comprising a first polypeptide which is autologous to a human subject coupled to a second polypeptide which is heterologous to the human subject, wherein the composition is capable of eliciting an immune response to an autologous antigen targeted for reduction or elimination.

In one embodiment, the autologous antigen is a cell-associated antigen. In one embodiment, the autologous antigen is a soluble antigen.

In another embodiment, the autologous antigen is selected from the group consisting of: CD64, sL-selectin, elastase, sCD16, CD46, TNF-α, sTNF-R75, sTNF-R55, TGF-β, CD40, CD154, lipoprotein (a), CD56, IL-10, IFN-γ, IL-2, IL-2R, CD45, IL-4, IgE, EGFR, TGF-β, CD54, sCD44 v5, and CD95.

In one embodiment, the autologous antigen is a tumor-associated antigen.

In another embodiment, the autologous antigen is expressed by a B cell.

In one embodiment, the first polypeptide and the second polypeptide are expressed as a fusion protein. In yet another embodiment, the fusion protein is dimeric.

In still another embodiment, the first polypeptide comprises at least a portion of a molecule selected from the group consisting of: CD79α, CD79β, CD20, and Ig.

In another embodiment, the second polypeptide comprises at least one T helper cell epitope

In one embodiment, the second polypeptide comprises at least a portion of an Fc region of an immunoglobulin molecule.

In yet another aspect, the invention pertains to compositions for targeting B cells in a subject comprising a first polypeptide, which is autologous to the subject, coupled to a second polypeptide, which is heterologous to the subject, wherein the first polypeptide comprises an immunogenic portion of a polypeptide expressed by a B cell in the subject and wherein the composition is capable of eliciting an immune response to an autologous B cell antigen in the subject.

In one embodiment, the autologous antigen is a cell-associated antigen. In another embodiment, the autologous antigen is a B cell tumor-associated antigen.

In one embodiment, the first polypeptide and the second polypeptide are expressed as a fusion protein. In one embodiment, the fusion protein is dimeric.

In one embodiment, the first polypeptide comprises at least a portion of a molecule selected from the group consisting of: CD79α, CD79β, CD20, and Ig.

In one embodiment, the second polypeptide comprises at least one T helper cell epitope In yet another embodiment, the second polypeptide comprises at least a portion of an Fc region of an immunoglobulin molecule.

In still another aspect, the invention pertains to compositions comprising a human polypeptide coupled to a polypeptide comprising at least a portion of a non-human immunoglobulin molecule. In one embodiment, the portion of the non-human immunoglobulin molecule is derived from the Fc portion of the immunoglobulin.

In another aspect, the invention pertains to nucleic acid molecules encoding a recombinant construct comprising a human polypeptide coupled to a non-human polypeptide, the construct being capable of eliciting an immune response against the human polypeptide in a human subject.

In yet another aspect, the invention pertains to a vector comprising the recombinant construct. In still another aspect, the invention pertains to a host cell comprising the vector of claim 43.

In one aspect, the invention pertains to methods of inducing an immune response against an autologous antigen in a subject, comprising: administering to the subject an immunogenic composition comprising a first, autologous polypeptide coupled to a second, heterologous polypeptide, such that an immune response is induced to an autologous antigen in the subject.

In yet another aspect, the invention pertains to a method of inducing an immune response against an autologous antigen associated with a disorder in a human subject, comprising: administering to the subject an immunogenic composition comprising a first, autologous polypeptide coupled to a second, heterologous polypeptide, such that an immune response is induced to an autologous antigen in the subject.

In one embodiment, a composition of the invention is administered to the subject more than once.

In one embodiment, an immune response is a T-cell dependent antibody response. In one embodiment, an antibody response comprises the production of antibodies of the IgG isotype that bind to the autologous antigen.

In one embodiment, an autologous antigen is a cell-associated antigen. In another embodiment, an autologous antigen is a soluble antigen.

In yet another embodiment, the autologous antigen is selected from the group consisting of: CD64, sL-selectin, elastase, sCD16, CD46, TNF-α, sTNF-R75, sTNF-R55, TGF-β, CD40, CD154, lipoprotein (a), CD56, IL-10, IFN-γ, IL-2, IL-2R, CD45, IL-4, IgE, EGFR, TGF-β, CD54, sCD44 v5, and CD95.

In one embodiment, a disorder is selected from the group consisting of: cancer, allergy, arthritis, atherosclerosis, graft rejection, and inflammatory disease.

In one embodiment, an autologous antigen is a tumor-associated antigen.

In another embodiment, an autologous antigen is expressed by a B cell.

In still another aspect, the invention pertains to a method of reducing the total amount or concentration of at least one class of antibody in the blood of a human subject comprising: administering to the human subject an immunogenic composition comprising a first, autologous polypeptide coupled to a second, heterologous polypeptide, wherein the first autologous polypeptide comprises at least a portion of a molecule expressed by a B cell of the human subject such that the total amount or concentration of at least one class of antibody in the blood of the human subject is reduced.

In yet another embodiment, a method of reducing the number or concentration of cells expressing a cell-associated, autologous antigen in a subject, comprising: administering to the subject an immunogenic composition, comprising a first, autologous polypeptide coupled to a second, heterologous polypeptide, such that the number or concentration of cells expressing the cell-associated, autologous antigen are reduced.

In one embodiment, the number or concentration of cells in the subject is reduced by at least about 50% relative to the number or concentration of cells in an untreated subject.

In one embodiment, the cells are B cells.

In yet another aspect, the invention pertains to methods of reducing the amount or concentration of a soluble autologous antigen present in a subject comprising: administering to the subject an immunogenic composition comprising a first, autologous polypeptide coupled to a second, heterologous polypeptide, such that an immune response is induced to a soluble autologous antigen in the subject.

### BRIEF DESCRIPTION OF THE DRAWINGS

FIG. 1 is a representation of a mouse (m)CD79b(Igβ)-human (h) IgG.Fc(γ4) fusion protein construct.

FIG. 2 panel A shows Igβ associated with Igα on B cells. Panel B displays the amino acid sequence of the mouse Igβ extracellular domain.

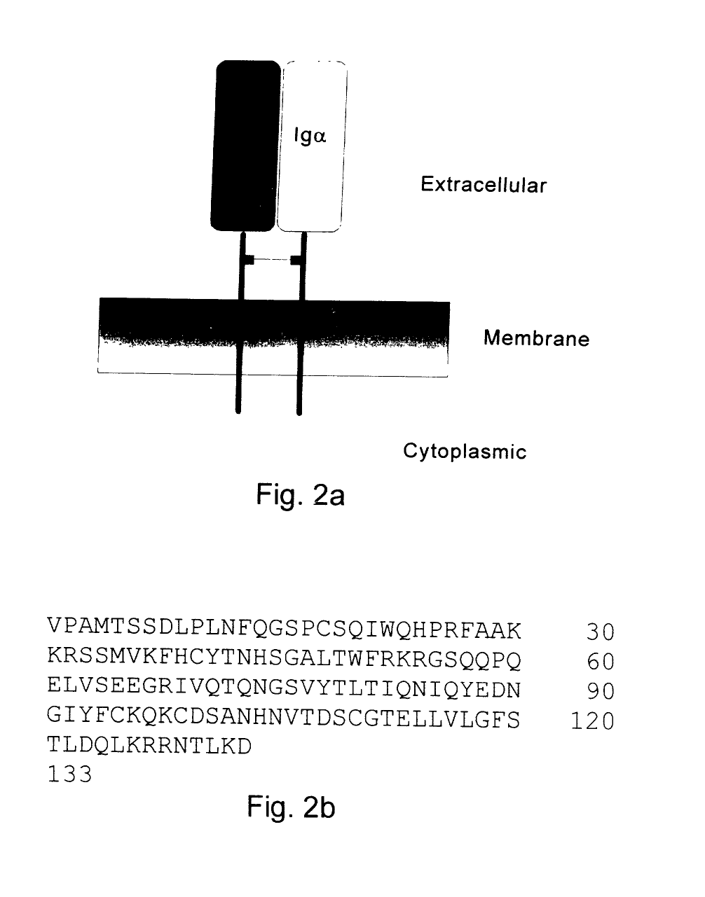

FIG. 3 is a histogram showing the sera derived from mice immunized with a fusion protein comprising mouse Igβ and human IgG.Fc can specifically stain BAL-17 lymphoma cell line as demonstrated using flow cytometry.

FIG. 4 panel A shows the characterization of mIgβ-hIgG.Fc using polyacrylamide gel electrophoresis and panel B shows the purification of the molecule by protein A affinity chromatography.

FIG. 5 shows the titer against autologous antigen after 4 immunizations. The screening antigen used was MBP-mIgβ at 5 ug/ml.

FIG. 6 panel A shows that sera from mice immunized with mIGβ-hIgG.Fc binds to BAL-17 cells, a mouse IgM producing B-cell lymphoma. Panel B shows that sera from mice immunized with hIgβ-hIgG.Fc binds less well to BAL-17 cells.

FIG. 7 shows changes in the B:T lymphocyte ratio. Panel A shows the results of immunization with hIgG.Fc and pane B shows the results of immunization with mIgβ-hIgG.Fc. FL1 is FITC conjugated rat anti-mouse K chain (staining for B lymphocytes) and FL2 is PE conjugated goat anti-mouse CD3 (staining for T lymphocytes).

FIG. 8 shows that the population of B lyymphocytes declines in Balb/c mice after repeated immunization with mIgGβ-hIgG.Fc as compared with hIgG.Fc.

### DETAILED DESCRIPTION

The instant invention provides compositions and methods for inducing immune responses to a target autologous antigen. The compositions of the invention comprise a first polypeptide coupled to a second polypeptide, wherein the second polypeptide is heterologous to a subject. The compositions are capable of eliciting an immune response against a target autologous antigen in a subject to which they are administered. The first polypeptide of the composition is autologous to the subject (or sufficiently homologous to an autologous polypeptide) such that an immune response to a target autologous antigen is induced in the subject to which the composition is administered. Using the instant compositions, active autoimmunity with unique specificity against a target autologous antigen can be induced.

Unlike the prior art methods of inducing autoimmunity, the instant compositions and methods are both specific and safe. After administration, the titer of induced autoantibodies returns to baseline levels, just as in the course of any antibody response. The lack of helper T cell determinants associated with the autologous target antigen as it is expressed naturally in the subject will not be sufficient to stimulate an immune response in the absence of an immunogenic composition of the invention. Only re-immunization with the same immunogenic composition will trigger the induction of a secondary auto-reactive immune response. In this manner, the extent of the autoreactivity can be controlled.

The claimed compositions and methods are useful in downmodulating the severity of a number of disorders or diseases, for example, cancer, allergy, arthritis, atherosclerosis, graft rejection, as well as autoimmune diseases.

Before further description of the invention, certain terms are, for convenience, defined here.

I. Definitions

As used herein, the term “immunogenic” with reference to a polypeptide or composition includes a polypeptide or composition that is capable of inducing an immune response.

As used herein, the term “immune response” includes T and/or B cell responses, i.e., cellular and/or humoral immune responses. In one embodiment, the claimed methods can be used to induce T helper cell responses. In another embodiment, the claimed methods can be used to induce humoral responses, e.g., T cell dependent antibody responses. An immune response can be either a primary or a secondary immune response. The immune response of a subject can be determined by, for example, assaying antibody production, immune cell proliferation, the release of cytokines, the expression of cell surface markers, cytotoxicity, etc. As used herein the term “anti-autologous target antigen immune response” refers to the immune response induced by a composition of the invention and that cross reacts with a target autologous antigen.

As used herein, the term “polypeptide(s)” refers to a peptide or protein comprising two or more amino acids joined to each other by peptide bonds or modified peptide bonds. “Polypeptide(s)” includes both short chains, commonly referred to as peptides, oligopeptides and oligomers and longer chains generally referred to as proteins. Polypeptides may contain amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to those of skill in the art. It will be appreciated that the same type of modification may be present in the same or varying degree at several sites in a given polypeptide. Also, a given polypeptide may contain many types of modifications. Modifications can occur anywhere in a polypeptide, including the peptide backbone, the amino acid side-chains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, glycosylation, lipid attachment, sulfation, gamma-carboxylation of glutamic acid residues, hydroxylation and ADP-ribosylation, selenoylation, sulfation, transfer-RNA mediated addition of amino acids to proteins, such as arginylation, and ubiquitination. See, for instance, Proteins—Structure And Molecular Properties, 2^{nd} Ed., T. E. Creighton, W. H. Freeman and Company, New York (1993) and Wold, F., Posttranslational Protein Modifications: Perspectives and Prospects, pgs. 1-12 in Posttranslational Covalent Modification Of Proteins, B. C. Johnson, Ed., Academic Press, New York (1983); Seifter et al., Meth. Enzymol. 182:626-646 (1990) and Rattan et al., Protein Synthesis: Posttranslational Modifications and Aging, Ann. N.Y. Acad. Sci. 663: 48-62 (1992). Polypeptides may be branched or cyclic, with or without branching. Cyclic, branched and branched circular polypeptides may result from post-translational natural processes and may be made by entirely synthetic methods, as well.

As used here, the term “autologous” with reference to a peptide (or the nucleic acid molecules which encode them) includes molecules which are not foreign to a subject (i.e., “self” polypeptides or nucleic acid molecules). The immune system can normally discriminate between self and “heterologous” (i.e., non-self or foreign) polypeptides and is non-reactive (e.g., tolerant) to self-antigens. By conjugation of a first polypeptide derived from (or having sufficient homology to) a target autologous antigen with a polypeptide derived from a heterologous antigen, the instant compositions and methods promote autoimmunity against the selected target autologous antigen. Preferably, autologous polypeptides are of therapeutic importance, i.e., modulation of cells bearing such polypeptides or a decrease in the level of such polypeptides will be of therapeutic benefit.

As used herein the term “autologous” with reference to a polypeptide of the invention includes self polypeptides and variants thereof (or the nucleic acid molecules that encode them) that are sufficiently homologous to (i.e., share sufficient nucleotide or amino acid sequence identity with) an autologous target polypeptide (or nucleic acid molecule encoding it) that they can promote an immune response to the target autologous antigen. Preferably, an autologous polypeptide is derived from the same species as the subject to which a composition comprising that polypeptide is administered. In one embodiment, autologous polypeptides can be replaced with polypeptides from another species that are sufficiently homologous to an autologous polypeptide. For example, if a polypeptide from a human and a polypeptide from a mouse contain highly conserved stretches of amino acids, peptides derived from the highly conserved mouse polypeptide may be functionally equivalent to the human polypeptide, in that the mouse polypeptide can promote the development of an anti-human target antigen immune response. In this example, because the polypeptide from the mouse is sufficiently homologous to the human polypeptide to promote an immune response to the human target autologous antigen in the subject, the mouse polypeptide is can be used in the subject constructs in place of an “autologous.” Polypeptides are said to be functionally equivalent to an autologous polypeptide if they are sufficiently homologous to an autologous polypeptide to promote an anti-target antigen immune response in a subject.

As used herein, the term “dimeric” includes soluble forms that are present as homodimers, i.e., as a unit comprised of two identical subunits which are joined together, e.g., by disulfide bonds.

Preferred polypeptides (and the nucleic acid molecules that encode them) are “naturally occurring.” As used herein, a “naturally-occurring” molecule refers to a molecule having an amino acid or a nucleotide sequence that occurs in nature (e.g., a natural polypeptide). In addition, naturally or non-naturally occurring variants of the polypeptides and nucleic acid molecules which retain the same functional activity, e.g., share immunologic cross-reactivity with a naturally occurring polypeptide are provided for. Such immunologic cross-reactivity can be demonstrated, e.g., by the ability of a variant to induce an immune response to a target autologous antigen or to bind to an antibody raised against the target autologous antigen. Such variants can be made, e.g., by mutation using techniques that are known in the art. Alternatively, variants can be chemically synthesized.

Polypeptides of the invention can be naturally occurring or can be variants of naturally occurring molecules. As used herein the term “variant(s)” includes nucleic acid molecules or polypeptides that differ in sequence from a reference nucleic acid molecule or polypeptide, but retain its essential properties. Changes in the nucleotide sequence of the variant may, or may not, alter the amino acid sequence of a polypeptide encoded by the reference nucleic acid molecule. Nucleotide or amino acid changes may result in amino acid substitutions, additions, deletions, fusions and truncations in the polypeptide encoded by a naturally occurring reference sequence. A typical variant of a polypeptide differs in amino acid sequence from a reference polypeptide. Generally, differences are limited so that the sequences of the reference polypeptide and the variant are closely similar overall and, in many regions, identical. A variant and reference polypeptide may differ in amino acid sequence by one or more substitutions, additions, and/or deletions in any combination.

A variant of a nucleic acid molecule or polypeptide may be naturally occurring, such as an allelic variant, or it may be a variant that is not known to occur naturally. Non-naturally occurring variants of nucleic acid molecules and polypeptides may be made to a reference nucleic acid molecule or polypeptide by mutagenesis techniques, by direct synthesis, and by other recombinant methods known to skilled artisans. Alternatively, variants can be chemically synthesized. For instance, artificial or mutant forms of autologous polypeptides which are functionally equivalent, (e.g., have the ability to bind to antibodies that bind to a reference polypeptide) can be made using techniques which are well known in the art.

Mutations can include, e.g., at least one discrete point mutation which can give rise to a substitution, or by at least one deletion or insertion. For example, mutations can also be made by random mutagenesis or using cassette mutagenesis. For the former, the entire coding region of a molecule is mutagenized by one of several methods (chemical, PCR, doped oligonucleotide synthesis) and that collection of randomly mutated molecules is subjected to selection or screening procedures. In the latter, discrete regions of a polypeptide, corresponding either to defined structural or functional determinants are subjected to saturating or semi-random mutagenesis and these mutagenized cassettes are re-introduced into the context of the otherwise wild type allele. In one embodiment, PCR mutagenesis can be used. For example, Megaprimer PCR can be used (O. H. Landt, 1990. Gene 96:125-128).

As used herein, the term “soluble” includes molecules, e.g., cytokines or hormones, which are not cell associated. As used herein, the term “cell-associated” includes those polypeptides which are not soluble, e.g., are attached to a cell via some mechanism (such as a transmembrane domain or a gpi-linkage).

As used herein, the term “extracellular domain” includes a portion of a molecule which, in the cell-associated form of the molecule, is extracellular.

As used herein, the term “adjuvant” includes agents which potentiate the immune response to an antigen. Adjuvants can be administered in conjunction with the described compositions to additionally augment an immune response.

As used herein, the term “dimeric” includes molecules that are present as homodimers or heterodimers, i.e., as a unit comprised of two identical subunits or two different subunits which are joined together, e.g., by disulfide bonds. As used herein, the term “multimeric” includes molecules having more than two subunits.

As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” or simply “expression vectors”. In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

As used herein, the term “host cell” is intended to refer to a cell into which a nucleic acid molecule of the invention, such as a recombinant expression vector of the invention, has been introduced. The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be a prokaryotic or a eukaryotic cell.

II. Compositions of the Invention

Polypeptides

The compositions of the invention comprise a first polypeptide having homology to an autologous target antigen, conjugated to a second heterologous polypeptide such that, upon introduction into a subject, an autoimmune response to an autologous target antigen is generated or enhanced in the subject. The first polypeptide can be autologous to the subject into which it will be introduced or can be functionally equivalent to such an autologous polypeptide. Alternatively, the compositions of the invention can comprise a nucleic acid molecule encoding a first polypeptide and a second nucleic acid molecule encoding a second heterologous polypeptide. Such a nucleic acid construct can induce an autoimmune response to an autologous target antigen sufficiently homologous to that first polypeptide encoded by the nucleic acid molecule.

Polypeptides (or the nucleic acid molecules that encode them) can be isolated from cells or tissue sources by an appropriate purification scheme using standard purification techniques. In another embodiment, a polypeptide can be produced by recombinant DNA techniques. Alternatively, polypeptides can be synthesized chemically using standard peptide synthesis techniques. In another embodiment, nucleic acid molecules encoding polypeptides can be synthesized for use in the claimed compositions. Whole cells expressing polypeptides can also be used in the compositions of the invention. For example, in one embodiment, a first polypeptide is expressed by a cell that is heterologous to the subject to which the composition will be administered.

The amino acid sequence of a polypeptide of the invention (or the nucleic acid sequence which encodes it) can be easily determined by one of ordinary skill in the art. There is a known and definite correspondence between the amino acid sequence of a particular protein and the nucleotide sequences that can code for the protein, as defined by the genetic code. Likewise, there is a known and definite correspondence between the nucleotide sequence of a particular nucleic acid molecule and the amino acid sequence encoded by that nucleic acid molecule, as defined by the genetic code.

An important and well known feature of the genetic code is its redundancy, whereby, for most of the amino acids used to make proteins, more than one coding nucleotide triplet may be employed. Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA molecule coding for a polypeptide (or a portion thereof) can be used to derive the amino acid sequence, using the genetic code to translate the DNA or RNA molecule into an amino acid sequence. Likewise, for any amino acid sequence, corresponding nucleotide sequences that can encode the polypeptide can be deduced from the genetic code (which, because of its redundancy, will produce multiple nucleic acid sequences for any given amino acid sequence). Thus, one of ordinary skill in the art, upon identifying a nucleotide sequence of a polypeptide should be considered to also to have the amino acid sequence encoded by the nucleotide sequence. Similarly, description and/or disclosure of an amino acid sequence herein should be considered to include description and/or disclosure of all possible nucleotide sequences that can encode the amino acid sequence.

In one embodiment, a polypeptide for use in the instant compositions is a naturally occurring polypeptide. In another embodiment, a polypeptide for use in the subject compositions does not occur naturally, but is a variant of a naturally occurring polypeptide. For example, variants of polypeptides can be generated by mutagenesis, e.g., discrete point mutation or truncation of a naturally occurring polypeptide.

In one embodiment, standard mutagenesis techniques, e.g., site-directed mutagenesis and PCR-mediated mutagenesis can be used to alter the sequence of a reference polypeptide (e.g., a polypeptide derived from an autologous target antigen) to obtain a functionally equivalent molecule. Such a variant may have enhanced properties, such as enhanced half life or enhanced immunogenicity. For example, in one embodiment, a conservative amino acid substitution can be made at one or more non-essential amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Alternatively, in another embodiment, mutations can be introduced randomly along all or part of a reference polypeptide coding sequence, such as by saturation mutagenesis, and the resultant mutants can be screened for their ability to encode polypeptides that bind to antibodies raised against the reference polypeptide sequence.

In one embodiment, variants of a polypeptide can be identified by screening combinatorial libraries of mutants, e.g., truncation mutants, of a naturally occurring polypeptide, e.g. by testing for their ability to bind to an antibody that reacts with a naturally occurring polypeptide. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential sequences is expressible as individual polypeptides, or alternatively, as a set of larger fusion proteins (e.g., for phage display).

There are a variety of methods which can be used to produce libraries of potential variants of a selected polypeptide from a degenerate oligonucleotide sequence. Chemical synthesis of a degenerate gene sequence can be performed in an automatic DNA synthesizer, and the synthetic gene can be ligated into an appropriate expression vector. Use of a degenerate set of genes allows for the provision, in one mixture, of all of the sequences encoding the desired set of potential polypeptide sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries comprising fragments of a protein coding sequence can be used to generate a variegated population of fragments for screening and subsequent selection of variants of a selected reference polypeptide. In one embodiment, a library of coding sequence fragments can be generated by treating a double stranded PCR fragment of a coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the polypeptide.

Several techniques are known in the art for screening gene products of combinatorial libraries made by point mutations or truncation, and for screening cDNA libraries for gene products having a selected property, e.g., the ability to bind to an antibody that binds to a reference sequence. Such techniques are adaptable for rapid screening of the gene libraries generated by the combinatorial mutagenesis of polypeptides. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants (Arkin and Yourvan (1992) Proc. Natl. Acad. Sci. USA 89:7811-7815; Delgrave et al. (1993) Protein Engineering 6(3):327-331).

In one embodiment, a polypeptide variant has at least about 95% amino acid identity with a naturally occurring polypeptide. In another embodiment, a polypeptide variant has at least about 90%, at least about 85%, at least about 80%, at least about 75% or at least about 70% or 60% amino acid sequence identity with a naturally occurring polypeptide. In one embodiment, a nucleic acid molecule that encodes a polypeptide variant encodes a polypeptide that has at least about 95% amino acid identity with a naturally occurring polypeptide. In another embodiment, a nucleic acid molecule that encodes a polypeptide variant having at least about 90%, at least about 85%, at least about 80%, at least about 75% or at least about 70% or 60% amino acid sequence identity with a naturally occurring polypeptide.

Alignments between polypeptides or nucleic acid molecules can be easily generated using techniques that are well known in the art. For example, to determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least about 50%, even more preferably at least about 60%, and even more preferably at least about 70%, 80%, 90%, 95%, or 100% of the length of the reference sequence.

The residues at corresponding positions are then compared and when a position in one sequence is occupied by the same residue as the corresponding position in the other sequence, then the molecules are identical at that position. The percent identity between two sequences, therefore, is a function of the number of identical positions shared by two sequences (i.e., % identity=# of identical positions/total # of positions×100). The percent identity between the two sequences is a function of the number of identical positions shared by the sequences, taking into account the number of gaps, and the length of each gap, which need to be introduced for optimal alignment of the two sequences. As used herein amino acid or nucleic acid “identity” is equivalent to amino acid or nucleic acid “homology”.

The comparison of sequences and determination of percent identity between two sequences can be accomplished using a mathematical algorithm. In a preferred embodiment, the percent identity between two amino acid sequences is determined using the GAP program in the GCG software package (available at http://www.gcg.com), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6.

The nucleic acid and protein sequences of the CTLA4 can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to CTLA4 nucleic acid molecules of the invention. BLAST protein searches can be performed with the XBLAST program, score=50, wordlength=3 to obtain amino acid sequences homologous to a reference polypeptide of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

In one embodiment, a polypeptide for use in a composition of the invention can comprise a complete protein or a fragment or derivative thereof. In one embodiment, a polypeptide for use in the claimed compositions comprises an entire protein. For example, in one embodiment, an autologous polypeptide comprises all or a part of the extracellular domain of a cell-associated molecule.

Alternatively, a polypeptide fragment or nucleic acid molecule encoding a polypeptide fragment can be used in the claimed compositions. An antigenic peptide fragment of a polypeptide typically comprises at least 8 amino acid residues (e.g., at least about 8, 10, 15, or 30 amino acid residues). Accordingly, the subject polypeptides can be of any length, e.g., from at least about 8 amino acids in length to a complete protein. Preferably, a polypeptide for use in the claimed compositions encompasses an epitope, e.g., a B cell and/or a T cell epitope.

In one embodiment, polypeptide epitopes for inclusion in the claimed compositions comprise domains that are located on the surface of an antigen in its native form. Such surface epitopes of a native molecule are preferably accessible for binding by an antibody. Surface epitopes can e.g., be derived from an extracellular domain of a cell-associated polypeptide or, e.g., from a hydrophilic regions of a soluble polypeptide.

In addition to polypeptides comprising only naturally-occurring amino acids, peptidomimetics can also be used. Peptide analogs are commonly used in the pharmaceutical industry as non-peptide drugs with properties analogous to those of the template peptide. These types of non-peptide compound are termed “peptide mimetics” or “peptidomimetics” (Fauchere, J. (1986) Adv. Drug Res. 15: 29; Veber and Freidinger (1985) TINS p.392; and Evans et al. (1987) J. Med. Chem 30:1229, which are incorporated herein by reference) and are usually developed with the aid of computerized molecular modeling.

Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biological or pharmacological activity), but have one or more peptide linkages optionally replaced by a linkage selected from the group consisting of: —CH2NH—, —CH2S—, —CH2—CH2—, —CH═CH— (cis and trans), —COCH2—, —CH(OH)CH2—, and —CH2SO—, by methods known in the art and further described in the following references: Spatola, A. F. in “Chemistry and Biochemistry of Amino Acids, Peptides, and Proteins,” B. Weinstein, eds., Marcel Dekker, New York, p. 267 (1983); Spatola, A. F., Vega Data (March 1983), Vol. 1, Issue 3, “Peptide Backbone Modifications” (general review); Morley, J. S., Trends Pharm Sci (1980) pp. 463-468 (general review); Hudson, D. et al., Int J Pept Prot Res (1979) 14:177-185 (—CH2NH—, CH2CH2—); Spatola, A. F. et al., Life Sci (1986) 38:1243-1249 (—CH2—S); Hann, M. M., J Chem Soc Perkin Trans I (1982) 307-314 (—CH—CH—, cis and trans); Almquist, R. G. et al., J Med Chem (1980) 23:1392-1398 (—COCH2—); Jennings-White, C. et al., Tetrahedron Lett (1982) 23:2533 (—COCH2—); Szelke, M. et al., European Appln. EP 45665 (1982) CA: 97:39405 (1982)(—CH(OH)CH2—); Holladay, M. W. et al., Tetrahedron Lett (1983) 24:4401-4404 (—C(OH)CH2—); and Hruby, V. J., Life Sci (1982) 31:189-199 (—CH2—S—); each of which is incorporated herein by reference. A particularly preferred non-peptide linkage is —CH2NH—.

Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), altered antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macromolecules(s) to which the peptidomimetic binds to produce the therapeutic effect. Derivitization (e.g., labelling) of peptidomimetics should not substantially interfere with the desired biological or pharmacological activity of the peptidomimetic.

Systematic substitution of one or more amino acids of an amino acid sequence with a D-amino acid of the same type (e.g., D-lysine in place of L-lysine) may be used to generate more stable peptides. In addition, constrained peptides comprising an autologous amino acid sequence or a substantially identical sequence variation may be generated by methods known in the art (Rizo and Gierasch (1992) Ann. Rev. Biochem. 61: 387, incorporated herein by reference); for example, by adding internal cysteine residues capable of forming intramolecular disulfide bridges which cyclize the peptide.

The amino acid sequences of polypeptides (or the nucleic acid sequences encoding them) appropriate for use in the instant compositions are known in the art and can be obtained, e.g., from a public or private database. Such amino acid sequences will enable those of skill in the art to produce polypeptides corresponding to peptide sequences and sequence variants thereof. Such polypeptides may be produced in prokaryotic or eukaryotic host cells by expression of polynucleotides encoding a peptide sequence, frequently as part of a larger polypeptide. Alternatively, such peptides may be synthesized by chemical methods. Methods for expression of polypeptides in recombinant hosts, chemical synthesis of polypeptides, and in vitro translation are well known in the art and are described further in Maniatis et al., Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91: 501; Chaiken I. M. (1981) CRC Crit. Rev. Biochem. 11: 255; Kaiser et al. (1989) Science 243: 187; Merrifield, B. (1986) Science 232: 342; Kent, S. B. H. (1988) Ann. Rev. Biochem. 57: 957; and Offord, R. E. (1980) Semisynthetic Proteins, Wiley Publishing, which are incorporated herein by reference).

Peptides can be produced, typically by direct chemical synthesis, and used in the claimed compositions. Peptides can be produced as modified peptides, with nonpeptide moieties attached by covalent linkage to the N-terminus and/or C-terminus. In certain preferred embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal-modifications such as amidation, as well as other terminal modifications, including cyclization, may be incorporated into various embodiments of the invention. Certain amino-terminal and/or carboxy-terminal modifications and/or peptide extensions to the core sequence can provide advantageous physical, chemical, biochemical, and pharmacological properties, such as: enhanced stability, increased potency and/or efficacy, resistance to serum proteases, desirable pharmacokinetic properties, and others.

In one embodiment, a polypeptide for use in a composition of the invention comprises the post-translation modifications, such as glycosylation.

Autologous Target Antigens and Homologous Polypeptides

Preferred autologous antigens for selection as targets of an anti-target immune response include: soluble autologous antigens which it would be desirable to reduce in quantity or amount or cell-associated autologous antigens present on cells which it would be desirable to reduce in number or percentage. Preferably, a directing the immune response to the target autologous antigen ameliorates a disease or disorder in a mammalian subject. Preferably, the subject is a human. Other subjects include, e.g., pets, exotic zoo animals, domesticated animals, etc.

An autologous target antigen can be selected, e.g., based on the desirability of lowering the amount or concentration of the target antigen or the number or percentage of a cell which expresses it. Exemplary target antigens are listed in Table 1.

A first polypeptide sufficiently homologous to that autologous target antigen is selected for incorporation into a composition of the invention such that an immune response against the first polypeptide will cross react with the target autologous antigen, e.g., as demonstrated by antibody binding.

In one embodiment, a first polypeptide has at least about 95% amino acid identity with an autologous target polypeptide. In another embodiment, a first polypeptide has at least about 90%, at least about 85%, at least about 80%, at least about 75% or at least about 70% or 60% amino acid sequence identity with an autologous target polypeptide. In one embodiment, a nucleic acid molecule that encodes a first polypeptide encodes a polypeptide that has at least about 95% amino acid identity with a an autologous target polypeptide. In another embodiment, a nucleic acid molecule that encodes a first polypeptide has at least about 90%, at least about 85%, at least about 80%, at least about 75% or at least about 70% or 60% amino acid sequence identity with an autologous target polypeptide.

In a preferred embodiment, a first polypeptide is derived from (or sufficiently homologous to) a target autologous antigen listed in Table 1 below.

| | Target autologous | |
|---|---|---|
| Disease | antigen | Reference(s) |
| Sickle cell disease | CD64, sL-selectin, | Lard, L. R. et al., J. |
| (SCD) | elastase, sCD16 | Leukoc. Biol. 66:411-5 |
| | | (1999) |
| Systemic lupus | CD46 | Kawano, M. et al., Clin. |
| erythematosus (SLE) | | Exp. Immunol. 116:542-6 |
| | | (1999) |
| CNS demyelinating | TNF-α | Kahn, M. A. et al., J. |
| diseases | | Neuroimmunol. 95:19-34 |
| | | (1999) |
| Inflammatory bowel | sL-selectin | Seidelin, J. B. et al., Am. J. |
| disease (IBD) | | Gastroenterol. 93:1854-9 |
| | | (1998) |
| Inflammatory bowel | TNF-α | Hanauer, S. B. et al., |
| disease (IBD) | | Aliment Pharmacol. Ther. |
| | | 13:16-22 (1999) |
| Renal cell carcinoma | sTNF-R75 | Elsasser-B., U. et al., |
| (RCC) | | Anticancer Res. 18:1883-90 |
| | | (1998) |
| Alcoholic liver | sTNF-R55 | aveau, S. et al., J. Hepatol. |
| cirrhosis | | 28:778-84 1998) |
| Acute alcoholic | sTNF-R75 | Naveau, S. et al., J. |
| hepatitis | | Hepatol. 28:778-84 (1998) |
| Rheumatoid arthritis | TNF-α | Alexopoulou, L. et al., Eur. |
| | | J. Immunol. 27:2588-92 |
| | | (1997) |
| | | Hanauer, S. B. et al., |
| | | Aliment Pharmacol. Ther. |
| | | 13:16-22 (1999) |
| Metastasis of | TGF-β | Moretti, S. et al., |
| melanoma | | Melanoma Res. 7:313-21 |
| | | (1997) |
| Carotid | CD40, | Mach, F. et al., Nature |
| atherosclerosis | CD40L (CD154) | 394:200-3 (1998) |
| Carotid | Lipoprotein (a) | Yamamoto, M. et al., |
| atherosclerosis | | Diabetes Care 20:829-31 |
| | | (1997) |
| Chronic myeloid | CD56 | Lanza, F. et al., Leukemia |
| leukemia (CML) | | 7:1570-5 (1993) |
| Rejection of heart | IL-10, | Furukawa, Y. et al., Am. J. |
| transplantation | IFN-γ | Pathol. 155:1929-39 (1999) |
| Rejection of kidney | IL-2, IL-2R | Gelder, T. et al., |
| transplantation | | Transplantation 60:248-52 |
| | | (1995) |
| Advanced acute | CD45 | Matthews, D. C. et al., |
| leukemia | | Blood 94:1237-47 (1999) |
| Allergy and | IL-4, | Barnes, P. J. et al., N. Engl. |
| chronic asthma | IgE | J. Med. 341:2006-8 (1999) |
| | | Milgrom, H. et al., N. Engl. |
| | | J. Med. 341:1966-73 (1999) |
| | | Chang, T. W. et al., |
| | | Biotechnol. 8:122-6 (1990) |
| Breast cancer | EGFR, TGF-β | Ma, L. et al., Int. J. Cancer |
| | | 78:112-9 (1998) |
| Intestinal | CD54 (ICAM-1) | Krieglstein, C. F. et al., Int. |
| inflammation | | J. Colorectal Dis. |
| | | 14:219-23 (1999) |
| | | Schneider, D. et al., Eur. |
| | | Neurol. 40:78-83 |
| Lupus nephritis | CD40L (CD154) | Kalled, S. L. et al., J. |
| | | Immunol. 160:2158-65 |
| | | (1998) |
| Metastasis of breast | sCD44 v5 | Lackner, C. et al., Breast |
| cancer | | Cancer Res. Treat. |
| | | 47:29-40 (1998) |
| Adult T-cell | Fas-R (CD95) | Kamihira, S. et al., Br. J. |
| Leukemia (ATL) | | Haematol. 99:858-65 |
| | | (1997) |

By selecting a first polypeptide that directs an immune response to a target autologous antigen listed in Table 1 (e.g., a polypeptide derived from one of the molecules listed in Table 1 or a variant thereof), an autoimmune response to the target autologous antigen is promoted or enhanced. In a preferred embodiment, the anti-target autologous antigen immune response serves to ameliorate a disease or disorder. Preferably, the disease or disorder is selected from those listed in Table 1.

In one embodiment, a target autologous antigen is a soluble polypeptide. In one embodiment, such a soluble polypeptide is secreted by an activated immune cell. For example, in one embodiment, a soluble polypeptide is a cytokine. Preferred cytokines for selection as a target autologous molecule include those that are important in growth and/or proliferation of immune cells (e.g., as autocrine growth factors), amplification of an ongoing immune response, or growth and/or proliferation of cancer cells. Exemplary soluble target autologous antigens include: TNF-α, TGF-β, IL-10, IFN-γ, IL-2, IL-4, IL-12, IL-3, EGF, PDGF, TGF-β, and the like.

In another embodiment, a target autologous antigen is a molecule normally secreted by an exocrine or endocrine cell. For example, an exemplary soluble target antigen is a hormone, e.g., a hormone used as a growth factor by a cancer cell, such as estrogen.

In another embodiment, a target autologous antigen is a cell-associated molecule that is membrane bound in its native form.

For example, in one embodiment, a target autologous antigen is expressed on the surface of a tumor cell. For example, a tumor-associated antigen can be expressed exclusively by a tumor cell or can be expressed at increased levels by a tumor cell, such that tumor cells are preferentially targeted by an immune response against the selected autologous target antigen.

In another embodiment, cell-associated target autologous antigen is expressed on the surface of an immune cell. As used herein, the term “immune cell” includes cells that are of hematopoietic origin and that play a role in the immune response. Immune cells include lymphocytes, such as B cells and T cells; natural killer cells; myeloid cells, such as monocytes, macrophages, eosinophils, mast cells, basophils, and granulocytes.

For example, in one embodiment a target autologous antigen is a cell surface receptor. In a preferred embodiment, a cell surface receptor is a cytokine receptor, a growth factor receptor, or an immune cell signal transducing receptor. Exemplary cytokine receptors include: TNF receptor, IL-4 receptor, IL-10 receptor, IL-12 receptor, and IL-2 receptor. Exemplary growth factor receptors include receptors for: EGF, PDGF, and bombesin. Exemplary immune cell signal transducing receptors include: CTLA4, CD3, membrane bound Ig (B Cell Receptor), TCR or associated molecules, CD79a (Igα), CD79b (Igβ), or FcR.

In another embodiment, a target autologous antigen is a membrane-bound molecule selected from the group consisting of: CD81, CD21, CD19, CD79, CD32, CD80, CD86, CD58, CD40, CD11a/CD18, CD22, CD45, CD28, CD2, CD4, CD8, CD154, CD54, CD43, and CD45.RO. Other exemplary sources for immune cell surface molecules can be found, e.g., in The Leucocyte Antigen Facts Book. (1997. Second Edition. Eds., Barlcay et al. Academic Press. Harcourt Brace & Co. Publishers. San Diego, Calif.)

In another embodiment, a target autologous antigen is selected from the group consisting of: CD64, sL-selectin, CD46, CD56, and CD95.

Preferably, a first polypeptide for use in the instant compositions is not derived from a class II molecule or a T cell receptor molecule.

In one embodiment, a target autologous antigen is expressed on the surface of B cells. Targeting of B cell antigens is desirable both when downmodulation of an unwanted immune response and upmodulation of a desired immune response would be advantageous. For example, as the producers of antibodies, B cells are excellent targets for elimination in the case of autoimmune or allergic subjects. Moreover, induction of immune response to a marker associated with a B cell malignancy would also be desirable.

In one embodiment, an autologous target antigen is expressed on cells other than B cells, but is expressed preferentially or at increased levels on B cells. In one embodiment, an autologous target antigen is a B cell activation or differentiation marker. In another embodiment, an autologous target antigen is expressed exclusively on B cells. For example, in one embodiment, a first polypeptide derived from a polypeptide selected from the group consisting of: Ig, CD79α, CD79β, CD20, and CD19.

In another embodiment, an autologous antigen for targeting is associated with or specific for a malignant B cell.

In one embodiment, an autologous B cell-associated molecule for targeting comprises at least a portion of an extracellular domain of a B cell surface molecule. Preferably, the target B cell surface molecule is a human molecule (expressed on a human B cell). For example, in a preferred embodiment, a first polypeptide comprises an extracellular domain of human CD79a (comprising at least a portion of amino acids 1-111 (Igα; numbering not inclusive of the signal sequence)) or human CD79b (Igβ; comprising at least a portion of amino acids 1-131 (numbering not inclusive of the signal sequence)); see, e.g., GenBank L32754 or L27587; Hashimoto, S. et al. 1994. Immunogenetics 40:287-295; Hashimoto, S. et al. 1994. Immunogenetics 40:145-149.

Heterologous Polypeptides

The subject compositions also comprise a second polypeptide which is heterologous to the subject into which the composition will be introduced. Heterologous polypeptides of the invention comprise at least one T cell epitope and, when conjugated to a first polypeptide having sufficiently homology to a target autologous antigen, serve to initiate or increase the immune response to the target autologous antigen in a subject.

A second, heterologous polypeptide can be expressed on a whole cell, can be an entire polypeptide, can be a portion or a fragment of a polypeptide, or can be an isolated T cell epitope.

Preferred heterologous polypeptides are mammalian in origin. Particularly preferred heterologous polypeptides are non-human in origin. Alternatively, heterologous polypeptides can be human in origin, but altered sufficiently such that they provoke an immune response in a human.

In one embodiment, a second, heterologous polypeptide is selected to alter (e.g., increase or decrease) the solubility, binding affinity, stability and/or valency (i.e., the number of epitopes per molecule) and/or increase the efficiency of protein purification of the composition.

Preferably, the second polypeptide comprises at least a portion of the Fc portion of an antibody molecule. (See e.g., U.S. Pat. No. 5,580,756, WO 97/28267; or U.S. Pat. No. 5,116,964, incorporated herein by reference). For example, as taught in the instant examples, a composition can be constructed by joining a first polypeptide to the C_{H}2-C_{H}3 regions of a heterologous immunoglobulin (Ig). In another embodiment, a second polypeptide can include the hinge-C_{H}2-C_{H}3 region of an Fc portion of an antibody.

A portion of an Fc domain of any isotype may be used as the second polypeptide in the claimed compositions, for example, Fc γ1, γ2, γ3, γ4, ε, μ, or α.

In one embodiment, a second polypeptide can comprise a variant of an Fc molecule, e.g., comprising mutations which modulate (e.g., up or downmodulate) effector functions, such as, complement and Fc receptor binding (Armour et al, 1999, Eur. J Immunol., 29:2613; Morgan et al, 1995, Immunology 86: 319; Lund et al, 1991, J.Immunol. 147:2657).

Methods of Coupling

A first polypeptide and a heterologous polypeptide of the invention can be linked using a variety of means known in the art to form the subject compositions. For example, first and second polypeptide can be functionally linked by chemical coupling, genetic fusion, noncovalent association or by other art-recognized methods.

In one embodiment, a first polypeptide and a second polypeptide can be directly linked. In another embodiment, a spacer sequence, e.g., of glycine and serine residues may be incorporated between the first polypeptide and a second polypeptide.

In one embodiment, a first polypeptide and a heterologous polypeptide of the invention are linked as a fusion protein. Fusion proteins can be produced by recombinant techniques may be secreted and isolated from a mixture of cells and medium containing the polypeptide or peptide. Alternatively, the polypeptide or peptide may be retained cytoplasmically and the cells harvested, lysed and the polypeptide isolated. A cell culture typically includes host cells, media and other byproducts. Suitable media for cell culture are well known in the art. Fusion proteins can be isolated from cell culture media, host cells, or both using techniques known in the art for purifying polypeptides and peptides. Techniques for transfecting host cells with appropriate constructs and purifying polypeptides and peptides are known in the art and are described in more detail below.

Preferably, a fusion protein of the invention is produced by standard recombinant DNA techniques. For example, DNA fragments coding for the different polypeptide sequences are ligated together in-frame in accordance with conventional techniques, for example employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide or an HA epitope tag). A nucleic acid molecule specifying a heterologous polypeptide can be cloned into an expression vector such that the fusion moiety is linked in-frame to polypeptide. A first polypeptide of the invention can be linked to a second polypeptide at its N or C terminus. In yet another embodiment, a fusion polypeptide further comprises a signal sequence at its N-terminus.

In one embodiment, a fusion protein of the invention is dimeric, i.e., is composed of two subunits. For example, cysteine residues, if present in a first or second polypeptide (either naturally or added by mutation) can form disulfide bonds resulting in polypeptide dimers. In one embodiment, a second, homologous polypeptide can comprise at least a portion of an Fc portion of an antibody with the cysteine residues can be conserved. In another embodiment, a first polypeptide is fused to at least a portion of the constant region of an IgM antibody to allow the formation of soluble multimers.

Methods of Making the Compositions

In one embodiment, the compositions of the instant invention are made recombinantly using nucleic acid manipulation. Techniques for nucleic acid manipulation are well known. (See, e.g., Sambrook et al., (1989); Ausubel et al. (1987) and in Annual Reviews of Biochemistry, 61:131-156 (1992)). Reagents useful in applying such techniques, such as restriction enzymes and the like, are widely known in the art and commercially available from a number of vendors.

Nucleic acid sequences encoding polypeptides for expression in the invention may be obtained using known procedures for molecular cloning and replication of the vector or plasmid carrying the sequences in a suitable host cell.

Nucleic acid sequences can be used in nucleic acid immunization procedures or can be used to produce polypeptides for use in the claimed compositions. Such nucleic acid molecules may also be produced in part or in total by chemical synthesis, e.g. by the phosphoramidite method described by Beaucage and Carruthers, Tetra. Letts. 22:1859-1862 (1981), or the triester method (Matteucci et al., J. Am. Chem. Soc. 103:3185 (1981)), and may be performed on commercial automated oligonucleotide synthesizers. A double-stranded fragment may be obtained from the single-stranded product of chemical synthesis either by synthesizing the complementary strand and annealing the strand together under appropriate conditions, or by synthesizing the complementary strand using DNA polymerase with an appropriate primer sequence.

To express a recombinant polypeptide, the natural or synthetic nucleic acid fragments coding for a desired sequence may be incorporated into vectors capable of introduction into and replication in a cell, e.g., a prokaryotic or eukaryotic cell. The vectors may be suitable for replication in a unicellular host, such as yeast or bacteria, but may also be introduced into cultured mammalian or plant or other eukaryotic cell lines, with or without integration within the genome. The vectors will typically comprise an expression system recognized by the host cell into which they are inserted, including the intended recombinant nucleic acid fragment encoding the desired polypeptide. The vectors will preferably also contain a selectable marker, i.e. a gene encoding a protein needed for the survival or growth of a host cell transformed with the vector. The presence of this gene ensures the growth of only those host cells which express the inserted nucleic acid of interest. Typical selection genes encode proteins that 1) confer resistance to antibiotics or other toxic substances, e.g. ampicillin, neomycin, methotrexate, etc.; b) complement auxotrophic deficiencies, or c)supply critical nutrients not available from complex media, e.g. the gene encoding D-alanine racemase for Bacilli. The choice of the proper selectable marker will depend on the host cell, and appropriate markers for different hosts are well known in the art. Such vectors may be prepared by means of standard recombinant techniques well known in the art (Sambrook et al., (1989); Ausubel et al., (1987)).

For gene transfer into the cells to express the selected molecules, nucleic acid may be directly introduced in the form of “naked” nucleic acid, e.g. by microinjection, electroporation, as calcium-phosphate-DNA gels, with DEAE dextran, or in encapsulated form, e.g. in vesicles such as liposomes, or in a suitable viral vector.

Vectors containing nucleic acid molecules for expression are preferably recombinant expression vectors in which high levels of gene expression may occur, and which contain appropriate regulatory sequences for transcription and translation of the inserted nucleic acid sequence. Regulatory sequences refers to those sequences normally associated (e.g. within 50 kb) of the coding region of a locus which affect the expression of the gene (including transcription of the gene, and translation, splicing, stability or the like, of the messenger RNA). A transcriptional regulatory region encompasses all the elements necessary for transcription, including the promoter sequence, enhancer sequence and transcription factor binding sites. Regulatory sequences also include, inter alia, splice sites and polyadenylation sites. An internal ribosomal entry site(IRES) sequence may be placed between recombinant coding sequences to permit expression of more than one coding sequence with a single promoter.

In addition, operational elements may include leader sequences, termination codons, and other sequences needed or preferred for the appropriate transcription and subsequent translation of the inserted nucleic acid sequences.

Secretion signals may also be included, whether from a native protein, or from a different secreted polypeptide of the same or related species. These signal sequences permit the molecule to enter cell membranes, and attain a functional conformation.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in activity assays, (e.g., direct assays or competitive assays described in detail below), or to generate antibodies specific for proteins, for example.

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the expression vector is a yeast expression vector. Examples of vectors for expression in yeast S. cerivisae include pYepSec1 (Baldari, et al., (1987) Embo J. 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, proteins can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid molecule of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

Alternatively, a polypeptide can be expressed in insect cells using baculovirus expression vectors. Baculovirus vectors available for expression of proteins in cultured insect cells (e.g., Sf 9 cells) include the pAc series (Smith et al., (1983) Mol. Cell Biol. 3:2156-2165) and the pVL series (Lucklow, V.A., and Summers, M.D., (1989) Virology 170:31-39).

In yet another embodiment, a nucleic acid molecule of the invention is expressed in mammalian cells using a mammalian expression vector. Examples of mammalian expression vectors include pMex-NeoI, pCDM8 (Seed, B., (1987) Nature 329:840) and pMT2PC (Kaufman et al. (1987), EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40.

Moreover, inducible regulatory systems for use in mammalian cells are known in the art, for example systems in which gene expression is regulated by heavy metal ions (see e.g., Mayo et al. (1982) Cell 29:99-108; Brinster et al. (1982) Nature 296:39-42; Searle et al. (1985) Mol. Cell. Biol. 5:1480-1489), heat shock (see e.g., Nouer et al. (1991) in Heat Shock Response, e.d. Nouer, L., CRC, Boca Raton, Fla., pp167-220), hormones (see e.g., Lee et al. (1981) Nature 294:228-232; Hynes et al. (1981) Proc. Natl. Acad. Sci. USA 78:2038-2042; Klock et al (1987) Nature 329:734-736; Israel & Kaufman (1989) Nucl. Acids Res. 17:2589-2604; and PCT Publication No. WO 93/23431), FK506-related molecules (see e.g., PCT Publication No. WO 94/18317) or tetracyclines (Gossen, M. and Bujard, H. (1992) Proc. Natl. Acad. Sci. USA 89:5547-5551; Gossen, M. et al. (1995) Science 268:1766-1769; PCT Publication No. WO 94/29442; and PCT Publication No. WO 96/01313). Accordingly, in another embodiment, the invention provides a recombinant expression vector in which a DNA is operatively linked to an inducible eukaryotic promoter, thereby allowing for inducible expression of a protein in eukaryotic cells.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding a protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) a protein. Accordingly, the invention further provides methods for producing a protein using the host cells of the invention. In one embodiment, the method comprises culturing the host cell of invention (into which a recombinant expression vector encoding a protein has been introduced) in a suitable medium such that a protein is produced. In another embodiment, the method further comprises isolating a protein from the medium or the host cell.

III. Administration of the Compositions of the Invention

The compositions of the invention can be administered to a subject either prophylactically or therapeutically.

A “therapeutically effective amount” of a composition of the invention is a dose sufficient to achieve a desired goal, e.g., to elicit an effective immune response against a target autologous antigen. A therapeutically effective amount can vary depending upon the peptide composition, the manner of administration, the stage and severity of the disease or disorder being treated, the weight and general state of health of the subject, and the judgment of the physician. Such amounts can generally range from about 0.01-10 mg per 70 kg to about 0.1-1.0 mg/70 kg per day. More commonly such amounts can vary from about 0.5-0.75 mg per 70 kg of body weight. Boosting dosages for subsequent administration can vary from about 0.1 mg to about 0.5 mg of polypeptide depending on the subject's response and condition.

Different subjects are expected to vary in responsiveness to such treatment. Dosages will vary depending on such factors as the individual's age, weight, height, sex, general medical condition, previous medical history, and immune status. Therefore, the amount or number of active molecules infused as well as the number and timing of subsequent infusions, is determined by a medical professional carrying out the therapy based on the response of the patient.

In one embodiment, the subject compositions will be administered repeatedly, e.g., in multiple doses, in order to promote a sufficient immune response to the target autologous antigen.

Toxicity and therapeutic efficacy of the subject compositions can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the in vitro assays and/or animal studies can be used in formulating a dosage range for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

After administration, the efficacy of the therapy can be assessed by a number of methods, such as assays that measure an immune response (e.g., anti-target antibody production, T cell proliferation, cytokine production, T cell cytotoxicity), the level of antigen being targeted; levels of cells bearing the autologous polypeptide, and/or clinical improvement of symptoms. Such assays are well know in the art. For example, levels of antibody against a target antigen can be measured using an ELISA assay, T cell proliferation can be measured using standard 3H thymidine uptake assays, T cell cytotoxicity can be measured using 51Cr release, cytokine production can be measured using ELISA, as can levels of soluble target polypeptide. Levels or numbers of cells bearing a target antigen can be quantified, e.g., using FACS analysis.

An increase in an immune response against a target autologous antigen, even if only short in duration, may be used as an indicator of efficacy of the therapy. For example, if the target molecule is a B cell marker, administration of a composition of the invention may initially enhance the level of antibodies that bind to the target molecule, but as the immune response progresses and B cells are eliminated, the antibody response against the target molecule will wane. One of ordinary skill in the art will be capable of assessing the relevant parameters to determine the appropriate course of therapy.

Efficacy may also be indicated by improvement in or resolution of the disease (pathologic effects), e.g., reduction or disappearance of an unwanted immune response, or improvement in or resolution of the disease (reduction in pathologic effects) associated with the unwanted immune response, improved graft tolerance, reduction in allergic reaction, etc. Thus, improvement in a specific condition for which treatment is being given can be monitored.

Standard methodologies can be used to assay either anti-autologous target immune responses or the status of the unwanted immune response to be reduced (e.g., by measuring T cell proliferation, cytokine production, numbers of activated T cells, antibody production, delayed type hypersensitivity, or antibody production) and are known in the art.

In one embodiment, it may be desirable to target multiple autologous antigens by administration of more than one composition. For example, a composition comprising a first peptide derived from a B cell molecule and an additional composition comprising a first peptide derived from a different B cell molecule could be administered to the same subject. In a second example, a composition comprising a first peptide derived from a B cell molecule and an additional composition comprising a first peptide derived from a T cell molecule could be administered to the same subject.

In one embodiment, the compositions of the instant invention can be administered in conjunction with an adjuvant. Exemplary adjuvants are known in the art and include: incomplete Freund's adjuvant, alum, aluminum phosphate, aluminum hydroxide, N-acetyl-muramyl-L-threonyl-D-isoglutamine, N-acetyl-nor-muramyl-L-alanyl-D-isoglutamine, N-acetylmuramyl-L-alanyl-D-isoglutaminyl-L-alanine-2-(1′2′-dipalmitoyl-sn-glycero-3-hydroxyphosphoryloxy)-ethylamine, RIBI (containing three components extracted from bacteria: monophosphoryl lipid A, trehalose dimycolate and cell wall skeleton), Freund's complete adjuvant, a cytokine or cytokines, or other immunostimulatory agent. Another exemplary adjuvant comprises squalene, pluronic L121 polymer and polysorbate in PBS (Kwak et al. 1992. New. Eng. J. Med. 327:1209).

IV. Pharmaceutical Compositions

The compositions of the instant invention can be administered as polypeptides or using “genetic immunization.” In one embodiment, a DNA expression vector encoding the composition is injected into the host animal, e.g., into the skin or into a muscle of the subject. The gene products are correctly synthesized and expressed by the subject. In one embodiment, DNA encoding a composition of the invention injected into muscles or delivered into the skin coated onto gold microparticles by a particle bombardment device, a “gene gun.” Genetic immunization has been shown to induce specific humoral responses and cellular immune responses (See, e.g., Mor et al. 1995. J. Immunol. 155:2039; Xu and Liew. 1995. Immunology. 84:173; Davis et al. 1994. Vaccine. 12:1503). Nucleic acid molecule can be administered via an intramuscular or intradermal route or the biolistic injection of DNA-coated gold particles into the epidermis can be accomplished with a gene gun that uses a particle accelerator or a compressed gas to inject the particles into the skin (Haynes et al. 1996. J. Biotechnol. 44:37)). Alternatively, nucleic acid vaccines can be administered by non-invasive means. For example, pure or lipid-formulated DNA can be delivered to the respiratory system or targeted elsewhere, e.g., Peyers patches by oral delivery of DNA (Schubbert. 1997. Proc. Natl. Acad. Sci. USA 94:961). Attenuated microorganisms can be used for delivery to mucosal surfaces. (Sizemore et al. 1995. Science. 270:29).

The compositions (e.g., polypeptide or nucleic acid) of the invention can be suspended in a any known carrier, e.g., a physiologically compatible pharmaceutical carrier (such as cell culture medium, physiological saline, phosphate-buffered saline, or the like) to form a physiologically acceptable, aqueous pharmaceutical composition. Suitable formulations are found, e.g., in Remington's Pharmaceutical Sciences (Mack Publishing Company, Philadlphia, Pa., 1985) Parenteral vehicles include sodium chloride solution, Ringer's dextrose, dextrose and sodium chloride, and lactated Ringer's. Other substances may be added as desired such as antimicrobials.

The compositions of the invention may be contacted with cells of a subject either in vivo or ex vivo. For example, the instant compositions may be introduced into the subject to be treated by using one of a number of methods of administration of therapeutics known in the art. For example, active molecules may be inoculated (with or without adjuvant) parenterally (including, for example, intravenous, intraperitoneal, intramuscular, intradermal, and subcutaneous), by ingestion, or applied to mucosal surfaces. Alternatively, the active molecules of the invention are administered locally by direct injection, e.g., into a cancerous lesion or graft site. “Inoculation” refers to administration of the active molecules of the invention to a subject.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition will be sterile and should be fluid to the extent that easy syringability exists. A composition will be stable under the conditions of manufacture and storage and can be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Routes of administration include epidermal administration including subcutaneous or intradermal injections. Transdermal transmission including iontophoresis may be used, for example “patches” that deliver product continuously over periods of time.

Mucosal administration of the active molecules of the invention is also contemplated, including intranasal administration with inhalation of aerosol suspensions. Suppositories and topical preparations may also be used. The methods of the invention contemplate the dosage of a sufficient amount or number of the active molecules to promote an anti-target immune response in a subject. The composition(s) may be introduced in at least one dose and either in that one dose or through cumulative doses are effective in promoting an anti-target immune response. The active molecules are administered in a single dose or in multiple, sequential doses.

The subject compositions can be included in a container, pack, or dispenser together with instructions for administration. The kit can further comprise a means for administering the active molecule of the invention, e.g., one or more syringes. The kit can come packaged with instructions for use.

V. Uses and Methods of the Invention

The compositions of the invention can be used to promote an autoimmune response against an autologous target antigen.

In one embodiment, the promotion of an anti-target autoimmune response may ultimately lead to the downmodualtion of an undesirable immune response. For example, if the autoimmune response results in autoreactivity against a target cell or cytokine involved in promoting an ongoing, unwanted immune response, the elimination of that target cell or cytokine via the specifically induced anti-target autoimmune response will result in the ultimate downmodulation of an undesirable immune response in the subject.

In another embodiment, e.g., where the target of the selectively induced autoimmune response is not a cell or molecule actively involved in the immune response (e.g., where the target is a hormone or tumor cell), then the administration of the constructs of the invention may result in the overall upregulation of an immune response is a subject, e.g., an anti-target hormone or tumor cell immune response.

Thus, although the compositions and methods of the invention upregulate an immune response to a selected autologous antigen, they ultimately can be used to either upmoduate or downmodulate a primary and secondary immune response to an antigen other than the autologous target. Similarly, while the immune response specific for the target autologous antigen is a T cell mediated humoral response, the subject compositions can be used to modulate different types of immune responses by (e.g., modulation of T cell responses by targeting a T cell; modulation of innate immunity by targeting macrophage; modulation of humoral immunity by targeting B cells).

Accordingly, in one aspect, the invention provides methods and compositions for preventing an undesirable immune response in a subject. A composition of the invention can be administered prior to the manifestation of symptoms for which modulation of the immune response would be beneficial, e.g., to prevent a disease or disorder. This type of protocol may be useful in the case of an inherited disorder. Alternatively, an unwanted immune response can be treated therapeutically (i.e., after symptoms have occurred) in order to ameliorate the unwanted response or delay its progression. In another embodiment, the invention provides a methods and compositions for inducing a desirable immune response in a subject e.g., an immune response to a cancer cell or a hormone. Again, a desirable immune response can be promoted before the onset of a disease or disorder or the subject compositions can be administered therapeutically.

Downmodulation of an Unwanted Immune Response

Downmodulation of an immune response is useful, e.g., in situations of tissue, skin and organ transplantation, in graft-versus-host disease (GVHD), allergy, or in autoimmune diseases (such as systemic lupus erythematosus, and multiple sclerosis). In the case of GVHD, the autologous polypeptide preferably a T cell marker and is autologous with respect to the donor of the T cells.

Blockage of immune cell function results in reduced tissue destruction. For example, in tissue transplants, rejection of the transplant is initiated through its recognition as foreign by immune cells, followed by an immune reaction that destroys the transplant. The administration of a composition of the invention that promotes an anti-target immune response against an immune cell or a cytokine, prior to or at the time of transplantation, can inhibit or reduce an immune response against the transplanted tissue.

The methods and compositions of the invention are also useful in treating autoimmune disease. Many autoimmune disorders are the result of inappropriate activation of immune cells that are reactive against self tissue and which promote the production of cytokines and autoantibodies involved in the pathology of the diseases. By inducing a specific anti-target immune response against, for example, B cells that promote autoimmunity, using the subject compositions, the unwanted autoimmune response may be downmodulate in order to reduce or eliminate disease symptoms.

Exemplary disorders that the instant methods can be used to treat include: inhibition of transplantation of organs or tissue, graft-versus-host diseases; rheumatoid arthritis, systemic lupus erythematosus, Hashimoto's thyroiditis, multiple sclerosis, myasthenia gravis, type I diabetes uveitis, juvenile-onset or recent-onset diabetes mellitus, posterior uveitis, allergic encephalomyelitis, glomerulonephritis, inflammatory and hyperproliferative skin diseases, psoriasis, atopical dermatitis, contact dermatitis, eczematous dermatitises, seborrhoeis dermatitis, urticaria, Lupus erythematosus, acne, Alopecia areata, keratoconjunctivitis, vernal conjunctivitis, pollen allergies, bronchial asthma, allergic asthma, intrinsic asthma, extrinsic asthma and dust asthma, chronic or inveterate asthma, late asthma and airway hyper-responsiveness, bronchitis, gastric ulcers, vascular damage caused by ischemic diseases and thrombosis, ischemic bowel diseases, inflammatory bowel diseases, and necrotizing enterocolitis.

Targeting immune cells or their cytokines can also be used therapeutically in the treatment of allergy and allergic reactions, e.g., to inhibit or downmodulate IgE production. Administration of a composition of the invention can be accompanied by exposure to antigen (e.g., allergen). Allergic reactions can be systemic or local in nature, depending on the route of entry of the allergen and the pattern of deposition of IgE on mast cells or basophils. Thus, an anti-target immune response can be promoted locally and/or systemically by administration of a composition of the invention.

To achieve optimal downmodulation of an unwanted immune response in a subject, it may be desirable to additionally administer other agents to the subject. For example, agents that block costimulatory function, e.g., soluble forms of B7-1, B7-2, or B7-1 and B7-2 or blocking antibodies against these antigens prior to or at the time of transplantation. Other downmodulatory agents that can be used include, for example, soluble forms of CTLA4, blocking antibodies against other immune cell markers or soluble forms of other receptor ligand pairs (e.g., agents that disrupt the interaction between CD40 and CD40 ligand (e.g., anti CD40 ligand antibodies)), antibodies against cytokines, fusion proteins (e.g., CTLA4-Fc), and immunosuppressive drugs, (e.g., rapamycin, cyclosporine A or FK506).

Upmodulation of a Desirable Immune Response

Upregulation of immune responses can be in the form of enhancing an existing immune response or eliciting an initial immune response against an antigen that it is desirous to reduce in amount or concentration. For example, an immune response to an autologous antigen that is not involved directly in an immune response, e.g., a tumor antigen or a hormone, can be stimulated in a naive subject or in a subject with an ongoing, low level of immunoreactivity against the antigen.

For example, in one embodiment, an immune response to a tumor cell antigen (e.g., sarcoma, melanoma, lymphoma, leukemia, neuroblastoma, carcinoma antigen) is promoted by administration of the subject compositions.

In certain instances, it may be desirable to further administer other agents that upregulate immune responses, for example, stimulatory forms of costimulatory molecules or cytokines can also be administered in order further augment an immune response against an autologous target antigen.

The practice of the present invention will employ, unless otherwise indicated, conventional techniques of cell biology, cell culture, molecular biology, genetics, microbiology, recombinant DNA, and immunology, which are within the skill of the art. Such techniques are explained fully in the literature. See, for example, Genetics; Molecular Cloning A Laboratory Manual, 2nd Ed., ed. by Sambrook, J. et aL (Cold Spring Harbor Laboratory Press (1989)); Short Protocols in Molecular Biology, 3rd Ed., ed. by Ausubel, F. et al. (Wiley, N.Y. (1995)); DNA Cloning, Volumes I and II (D. N. Glover ed., 1985); Oligonucleotide Synthesis (M. J. Gait ed. (1984)); Mullis et al. U.S. Pat. No: 4,683,195; Nucleic Acid Hybridization (B. D. Hames & S. J. Higgins eds. (1984)); the treatise, Methods In Enzymology (Academic Press, Inc., N.Y); Immunochemical Methods In Cell And Molecular Biology (Mayer and Walker, eds., Academic Press, London (1987)); Handbook Of Experimental Immunology, Volumes I-IV (D. M. Weir and C. C. Blackwell, eds. (1986)); and Miller, J. Experiments in Molecular Genetics (Cold Spring Harbor Press, Cold Spring Harbor, N.Y. (1972)).

The contents of all references, pending patent applications and published patents, cited throughout this application are hereby expressly incorporated by reference. Each reference disclosed herein is incorporated by reference herein in its entirety. Any patent application to which this application claims priority is also incorporated by reference herein in its entirety.

The invention is further illustrated by the following examples, which should not be construed as further limiting.

### EXAMPLES

### Example 1

Induction of Autoimmunity Using an Autologous Polypeptide-Foreign Polypeptide Construct

In this example, surface molecules on autologous B cells were targeted. Specifically, Igβ was chosen for immunological intervention in order to regulate B cell activity by elimination of B-cells. Ig is a plasma membrane phosphoprotein expressed exclusively by B-lymphocytes. It is disulfide-linked and membrane-bound (Kooten, et al., 1997, Clin. Exp. Immunol., 110:509-515). Using a mouse model auto-antibodies specific for Ig were induced by immunization with of mice with the extracellular domain of autologous, murine Igβ linked to a heterologous, human IgG.Fc domain. The anti self-antigen antibodies induced in the mice mimic the activity of passively administered anti-self antigen antibody and function to down-regulate B cells.

Construction of Representative Expression Vectors

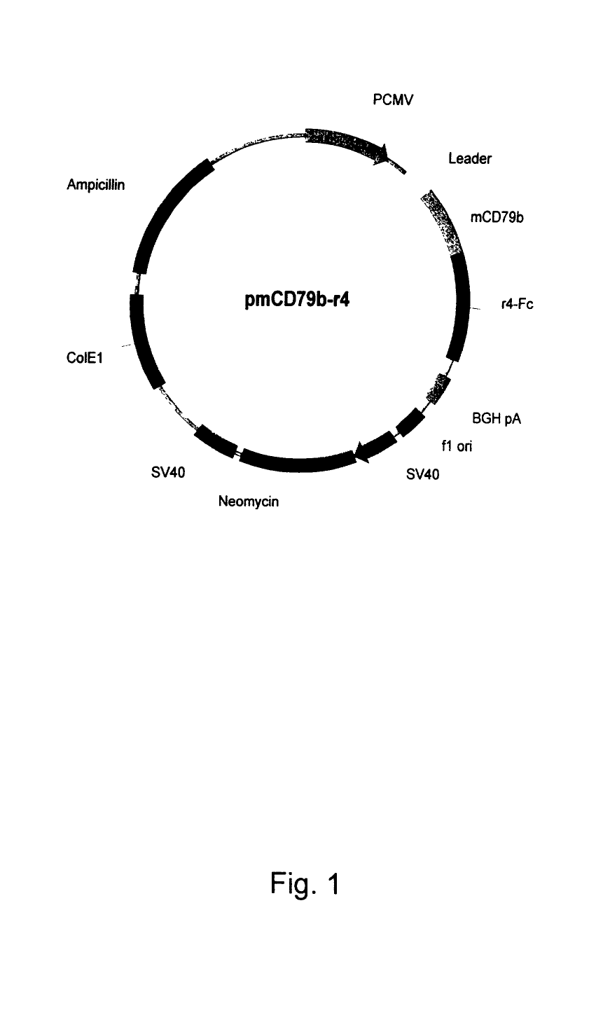

The vector pcDNA3 containing a neomycin gene was used as the expression cassette in this example. The fusion genes were inserted into the vector using a 5′ end HindIII site and a 3′ end EcoRI cleavage site. The fusion genes comprised the extracellular segment of mouse Igβ (containing 133 amino acids) and the CH2—CH3 domains of human IgG.Fc. In this case the mouse Igβ gene segment was fused through a peptide linker upstream from the gene segment of CH2—CH3 domains of human IgG.Fc. The mouse Igβ gene segment can be placed upstream of human IgG.Fc fragment, e.g., by making use of KpnI and BamHI cleavage sites, and can be joined together by an peptide linker (e.g., the immunologically inert peptide linker of 16 amino acids as illustrated in FIG. 1). Alternatively, the peptide linker between both segments can comprise an enterokinase cleavage site (consisting of 5 amino acids). Such a construct can be useful for purification of constructs after enterkinase cleavage.

Transfection and Selection of Candidate Clones Expressing High Level of Fusion Protein

Transfection was performed by liposome methods (Clontech, USA) according to manufacture's standard procedures. CHO-dhfr(−) cells from Culture Collection Research Center (Taiwan) were cultured in IMDM containing 10% fetal bovine serum and antibiotics; penicillin and streptomycin, all were obtained from GIBCO, New York. Stable cell lines were made by seeding 1×10⁶ CHO cells in a 10-cm petri dish in growth media without serum. Following up to 24-hour of incubation, each dish was transfected with 10 μg of plasmid followed by another 6-hour incubation. Cells were then transferred to the growth medium containing serum and incubated for further 48-hours.

The cells were reseeded to 96-well plates at a cell density of 10⁴ per well and transferred to growth medium containing serum and the toxin G418 (0.6 mg/ml) for selection. After 10-21 days, resistant colonies were picked and assayed for human IgG.Fc protein production. Determination of the protein levels was accomplished using standard ELISA. The methodologies for these assays are well described in the art.

Purification Scheme by Affinity Chromatography

The constructs were purified using art-recognized methods. Candidate cell lines showing higher protein production than other cell lines were propagated in 24-well and then expanded into 6-well dishes. Ultimately, cells were expanded into roller bottles. For growth of cells, bovine IgG was removed by passing media through Protein A-Sepharose 4B gels to avoid the cross contamination of the IgG.Fc fusion protein constructs during the purification process. The fusion proteins were then purified from batch culture medium using Protein A-Sepharose 4B gel affinity chromatography. Routinely, 2 to 10 mg of fusion protein is obtained from 1 liter of serum-containing media.

Protein Characterizations

The human IgG.Fc portion of the fusion constructs were previously characterized during the ELISA protocols for screening of transfectants. The molecular size of recombinant fusion proteins expressed by transfactants was determined by SDS-PAGE. The protein markers with known molecular sizes were used as standards.

Mouse Immunization

Inbred Balb/c mice aged from 6 to 8 months were bought from National Animal Center (Taiwan). Three groups of Balb/c mice (n=15, 8 and 5) were used in this example. The first group of mice was immunized with the mouse Ig-human IgG.Fc fusion construct at a dosage of 50 μg per mouse by subcutaneous injection. The second group was immunized with human IgG proteins, which were purified form human sera (Cappel Laboratories). The dosage for immunization of mice with human IgG was the same as for the mouse Igβ-human IgG.Fc fusion constructs. The third group served as a control group and was not injected.

The immunization protocol consisted of four injections and collection of blood of each mouse 7-day after each immunization. For the first immunization, complete Freund's adjuvant was used to emulsify the immunogen. For subsequent immunizations, immunogens were emulsified with incomplete Freund's adjuvant prior to immunization.

Detecting Immune Responses

Blood collected from the mice was used for the evaluation of the immune responses of mice. Microtiter plates pre-coated with human IgG or the extracellular domain of mouse Igβ (linked to maltose binding protein (MBP) expressed in an E. coli system) as antigens for ELISA to determine the antibody titers in the sera of injected mice. The relative average titers of each mouse group are listed below in Table I:

TABLE I
Titers of sera obtained from immunized mice against the following
fusion proteins after four immunizations

| | MBP-mouse Igβ segment | Human IgG |
|---|---|---|
| Group I immunized with mouse | + | + |
| Igβ segment and human IgG.Fc | | |
| fusion protein (n = 15) | | |
| Group II immunized with human | − | + |
| IgG proteins | | |
| (n = 5) | | |
| Group III; Negative control | − | − |
| (n = 8) | | |

* The symbols represent the strength of mouse serum titers assayed by ELISA.
“+” is the average titer of the mouse group in dilution folds higher than 2,500.
“−” is no titer observed in the mouse group.

Determination of the Percentages of B Cells in Total Lymphocytes in Immunized Mice

Measurements of the depletion of B cells in the three groups of immunized mice was performed using flow cytometry. The B- and T-cell staining protocols are described as below. Three to 5 drops of peripheral blood were taken from the tail vein of each mouse and the red blood cells were removed using lysing solution (Tris-HCl) on ice for 20 min. The B- and T-cells were simultaneously double-stained with 10 μl of FITC labeled rat anti mouse kappa and 0.5 μl of PE labeled rat anti mouse CD3 (Pharmingen) for 20 min. Cells were blocked and washed with 2 ml of HBSS containing 5% fetal bovine serum (HBSS-5%FBS). The stained B- and T-lymphocytes were placed in 0.5 ml of HBSS-5% FBS. The fluorescence-labeled cells were measured using two color flow cytometry. The average percentages of B-cells in total B- and T-lymphocytes in each mouse group are summarized in Table II:

TABLE II
The Average percentages of B-lymphocytes in blood of mice
immunized four-times with mouse Igβ segment-human IgG.Fc
fusion protein and human IgG proteins

| | Percentages of B-cells | Percentages of T-cells |
|---|---|---|
| | in overall population | in overall population |
| No. of mice group | of B- and T-cells (%) | of B- and T-cells (%) |
| Group I immunized | 35.0* | 65.0 |
| with mouse Igβ | | |
| segment and human | | |
| IgG.Fc fusion protein | | |
| (n = 15) | | |
| Group II immunized | 66.0* | 34.0 |
| with human IgG | | |
| proteins | | |
| (n = 5) | | |
| Group III; Negative | 52.3* | 47.7 |
| control | | |
| (n = 8) | | |

*The P values of the Group I as compared to Group II and Group III were calculated by t-test in values of 3.34 × 10⁻⁷ and 1.11 × 10⁻⁵, respectively. The P value < 0.05 represents the means of these two groups showing significant variations under 95% of confidence.

The percentages of B-cells in overall lymphocyte population in Group I declined to a level of 35.0%, lower than that seen in group II (66.0%) and group III (52.3%). The results indicate the autologous mouse Ig/human IgG.Fc fusion protein constructs were capable of inducing the depletion of B-cells to at least about a 50% of the level seen in control mice.

### Example 2

Binding of Native Ig Molecules on BAL-17 Lymphoma Cell Line by Mouse Sera

Sera from mice immunized using constructs of the invention was found to recognize and bind to the native Ig molecules on BAL-17 lymphoma cells.

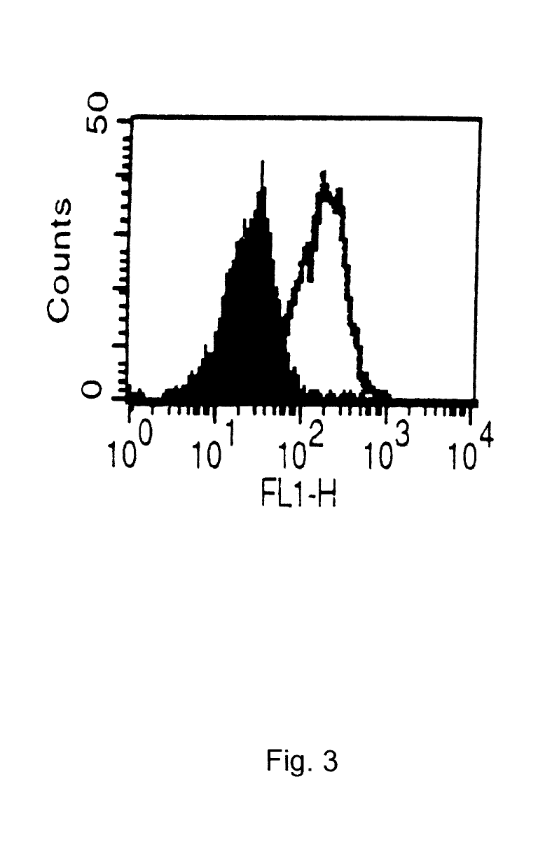

Sera from representative mice of each group immunized in Example 1 were utilized to stain the BAL-17 cells on ice for 20 min. Following washing and blocking of the cells with 2 ml of HBSS containing 5% fetal bovine serum (HBSS-5% FBS), the cells were stained with a secondary FITC labeled rat anti-mouse IgG2a monoclonal antibody (Pharmingen). The cells were then suspended in 0.5 ml of HBSS-5% FBS flow cytometry was performed as described above. The histograms of the cell-staining results are depicted in FIG. 3.

### Example 3

Induction of Specific Autoantibodies Against CD79a by Immunization with Heterologous CD79a Fused to Heterologous IgG1.Fc

Mice were immunized with the extracellular portion of human CD79α (Igα), coupled with human IgG1.Fc as a dimeric fusion protein. The immunized mice make antibodies against autologous Igβ. This is true despite the fact that there is only 55% amino acid sequence identity between the extracellular domain of human and mouse Igα. The sera from mice immunized with human Fc-Igα contained IgG that was reactive with mouse Igα by ELISA. The sera could stain cells of a mouse IgM-expressing B cell line, BAL17, which expresses Igα, as shown by fluorescence flow cytometry. In addition, the levels of total B cells in the immunized mice declined after the elicitation of autoantibodies against Igα. Analogous immunization with recombinant human Fc-Igβ fusion protein constructs lead to a much weaker response to mouse Igβ, when compared to that for Igα, although human and mouse Igβ also share a similar degree of amino acid sequence identity in their extracellular domains. Hydrophilicity and immunogenicity plots show that several consecutive, potentially immunogenic segments sharing similar or nearly identical amino acid sequences between human and mouse Igα, whereas this is not the case for Igβ.

### Example 4

Effect of mIgβ-hIgG.fc or mCD20-hIgG.Fc on Levels of B Cells and their Activities

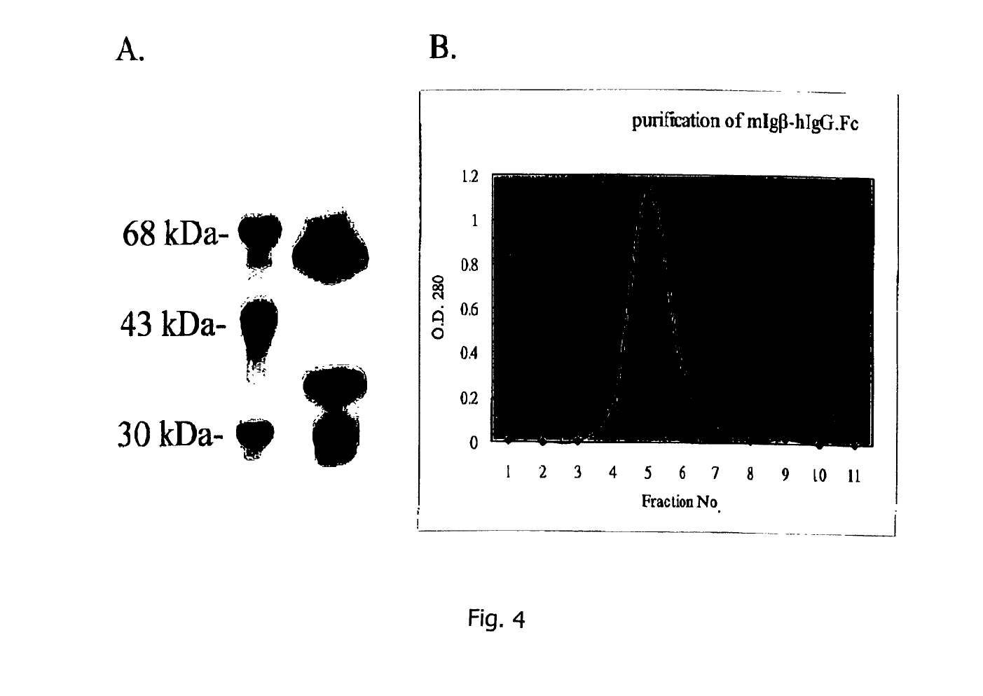

Mouse Igβ-human IgG.Fc and mouse CD20-human IgGFc fusion proteins were made. The constructs were inserted into vectors and then transfected into CHO.K1 cells using standard methods. After G418 selection and screening, the stable transfectants with a high level of protein expression were selected and maintained as a kind of cell line for long-term operation. The expressed mouse Igβ-human IgG.Fc proteins (mIgβ-hIgG.Fc) were purified by running the cultured medium through a protein-A affinity column (protein-A can specifically bind with the Fc portion of fusion proteins) (FIG. 4B) After identification and characterization, the purified proteins were used as compositions for administration (FIG. 4A).

15 Balb/c mice were immunized with mIgβ-hIgG.Fc proteins independently, once per two weeks. One week after every boost, about 100 ml of blood was collected from each immunized mouse for the quantification of B-/T-lymphocyte ratio changes. The anti-sera were also prepared for the identification of titer specificity.

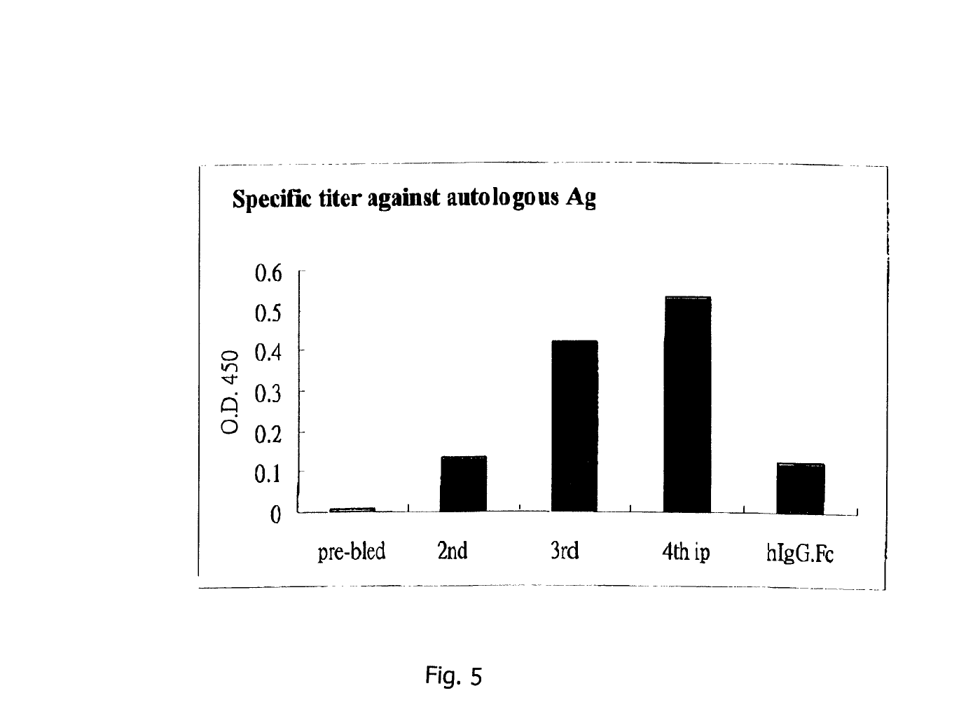

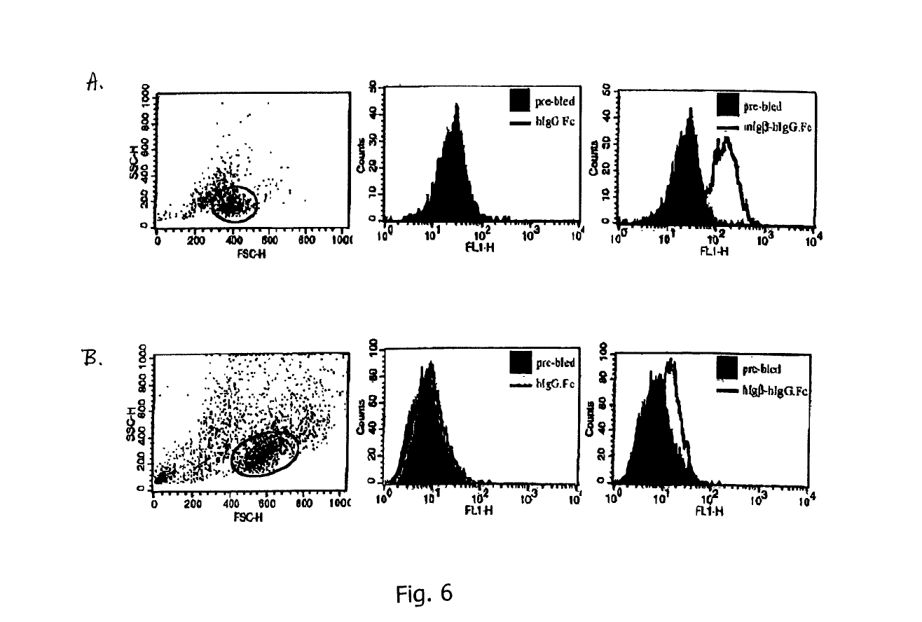

The titer specificity of anti-sera was tested using ELISA. The MBP fused mouse Igβ proteins, as a screening antigen, were purified from an E. coli protein expression system and coated on ELISA plates at a final concentration of 5 ug/ml, 50 ml per well. The specific titer against autologous mouse Igβ is shown by ELISA (FIG. 5). The antisera stains the cell surface of a mouse IgM expressing B cell line, BAL17, which expresses Igβ, as shown by fluorescence flow cytometry (FIG. 6A). Analagous immunization with recombinant human Igβ-hIgG.Fc fusion protein led to a much weaker response to BAL-17 (FIG. 6B).

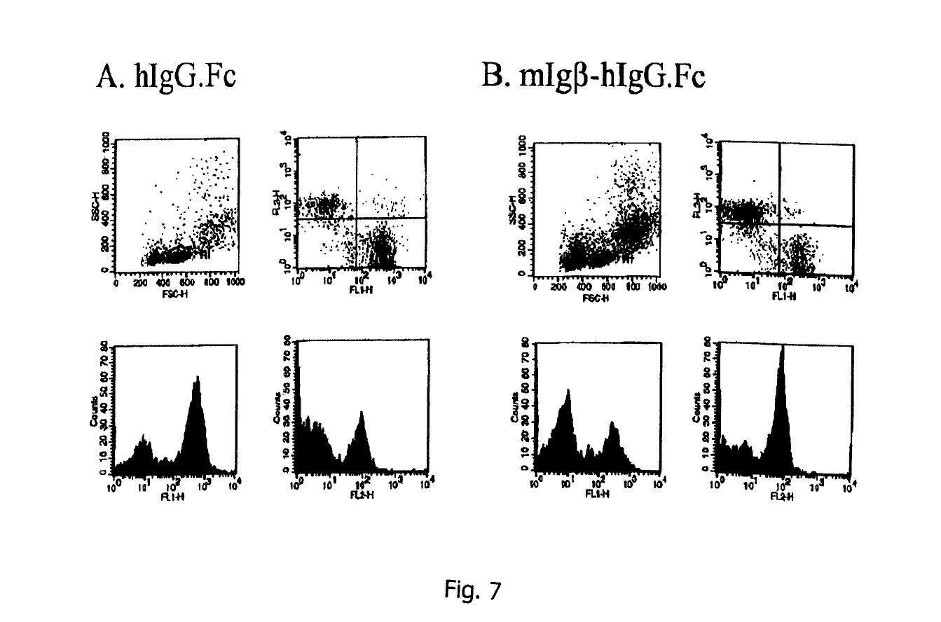

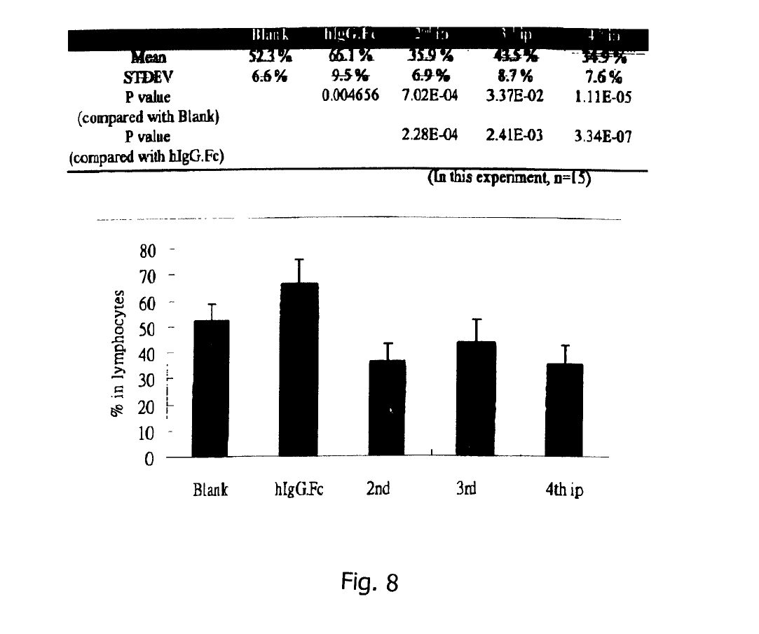

The levels of total B cells in the immunized mice declined after the elicitation of autoantibodies against Igβ (FIG. 7B), compared to immunization with hIgG.Fc alone (FIG. 7A. The P value of the B-lymphocyte ratio changes is less than 0.05, and the regulatory roles of this protein vaccine on B cell activity could be identified (FIG. 8). Similar results have been achieved after immunization with CD20-hIgG.Fc proteins.

The results presented in this Example show that specific autoimmune antibodies against targeted autologous antigen expressed by B cells are induced using these constructs and that they have an effect on the levels of B cells and on their activities.

Equivalents

Those skilled in the art will recognize, or be able to ascertain using no more than routine experimentation, numerous equivalents to the specific polypeptides, nucleic acids, methods, assays and reagents described herein. Such equivalents are considered to be within the scope of this invention and are covered by the following claims.

What is claimed is:

1. An immunogenic composition, comprising: a first polypeptide coupled to a second polypeptide, wherein the second polypeptide is heterologous to a subject, the composition being capable of eliciting an immune response against an autologous antigen in the subject.

2. An immunogenic composition, comprising: a first polypeptide, wherein the first polypeptide is sufficiently homologous to an autologous polypeptide in a subject, coupled to a second polypeptide, wherein the second polypeptide is heterologous to the subject, the composition being capable of eliciting an immune response against an autologous antigen in the subject.

3. An immunogenic composition, comprising: a first polypeptide, which is autologous to a subject, coupled to a second polypeptide, which is heterologous to the subject, the composition being capable of eliciting an immune response against an autologous antigen in the subject.

4. The composition of claim 3, wherein the subject is a human.

5. The composition of claim 3, wherein the autologous antigen is a cell-associated antigen.

6. The composition of claim 5, wherein the autologous antigen is a cell surface receptor.

7. The composition of claim 3, wherein the autologous antigen is a soluble antigen.

8. The composition of claim 7, wherein the autologous antigen is a cytokine or a hormone.

9. The composition of claim 3, wherein the autologous antigen is selected from the group consisting of: CD64, sL-selectin, elastase, sCD16, CD46, TNF-α, sTNF-R75, sTNF-R55, TGF-β, CD40, CD154, lipoprotein (a), CD56, IL-10, IFN-γ, IL-2, IL-2R, CD45, IL-4, IgE, EGFR, TGF-β, CD54, sCD44 v5, and CD95.

10. The composition of claim 3, wherein the autologous antigen is a tumor-associated antigen.

11. The composition of claim 3, wherein the autologous antigen is expressed by a B cell.

12. The composition of claim 11, wherein the autologous antigen is expressed specifically by B cells.

13. The composition of claim 11, wherein the autologous antigen is expressed specifically by activated B cells.

14. The composition of claim 3, wherein the first polypeptide and the second polypeptide are expressed as a fusion protein.

15. The composition of claim 14, wherein the fusion protein is dimeric.

16. The composition of claim 3, wherein the first polypeptide and the second polypeptide are coupled via a chemical linkage.

17. The composition of claim 3, wherein the first polypeptide comprises at least a portion of a molecule selected from the group consisting of: CD79α, CD79β, CD20, and Ig.

18. The composition of claim 3, wherein the second polypeptide comprises at least one T helper cell epitope.

19. The composition of claim 3, wherein the second polypeptide comprises at least a portion of an Fc region of an immunoglobulin molecule.

20. A composition comprising a first polypeptide which is autologous to a human subject coupled to a second polypeptide which is heterologous to the human subject, wherein the composition is capable of eliciting an immune response to an autologous antigen targeted for reduction or elimination.

21. The composition of claim 20, wherein the autologous antigen is a cell-associated antigen.

22. The composition of claim 20, wherein the autologous antigen is a soluble antigen.

23. The composition of claim 20, wherein the autologous antigen is selected from the group consisting of: CD64, sL-selectin, elastase, sCD16, CD46, TNF-α, sTNF-R75, sTNF-R55, TGF-β, CD40, CD154, lipoprotein (a), CD56, IL-10, IFN-γ, IL-2, IL-2R, CD45, IL-4, IgE, EGFR, TGF-β, CD54, sCD44 v5, and CD95.

24. The composition of claim 20, wherein the autologous antigen is a tumor-associated antigen.

25. The composition of claim 20, wherein the autologous antigen is expressed by a B cell.

26. The composition of claim 20, wherein the first polypeptide and the second polypeptide are expressed as a fusion protein.

27. The composition of claim 26, wherein the fusion protein is dimeric.

28. The composition of claim 20, wherein the first polypeptide comprises at least a portion of a molecule selected from the group consisting of: CD79α, CD79β, CD20, and Ig.

29. The composition of claim 20, wherein the second polypeptide comprises at least one T helper cell epitope

30. The composition of claim 20, wherein the second polypeptide comprises at least a portion of an Fc region of an immunoglobulin molecule.

31. A composition for targeting B cells in a subject comprising a first polypeptide, which is autologous to the subject, coupled to a second polypeptide, which is heterologous to the subject, wherein the first polypeptide comprises an immunogenic portion of a polypeptide expressed by a B cell in the subject and wherein the composition is capable of eliciting an immune response to an autologous B cell antigen in the subject.

32. The composition of claim 31, wherein the autologous antigen is a cell-associated antigen.

33. The composition of claim 31, wherein the autologous antigen is a B cell tumor-associated antigen.

34. The composition of claim 31, wherein the first polypeptide and the second polypeptide are expressed as a fusion protein.

35. The composition of claim 34, wherein the fusion protein is dimeric.

36. The composition of claim 31, wherein the first polypeptide comprises at least a portion of a molecule selected from the group consisting of: CD79α, CD79β, CD20, and Ig.

37. The composition of claim 31, wherein the second polypeptide comprises at least one T helper cell epitope

38. The composition of claim 31, wherein the second polypeptide comprises at least a portion of an Fc region of an immunoglobulin molecule.

39. A composition comprising human polypeptide coupled to a polypeptide comprising at least a portion of a non-human immunoglobulin molecule.

40. The composition of claim 39, wherein the portion of the non-human immunoglobulin molecule is derived from the Fc portion of the immunoglobulin.

41. A nucleic acid molecule encoding a recombinant construct comprising a human polypeptide coupled to a non-human polypeptide, the construct being capable of eliciting an immune response against the human polypeptide in a human subject.

42. A vector comprising the recombinant construct of claim 41.

43. A host cell comprising the vector of claim 42.

44. A method of inducing an immune response against an autologous antigen in a subject, comprising: administering to the subject an immunogenic composition comprising a first, autologous polypeptide coupled to a second, heterologous polypeptide, such that an immune response is induced to an autologous antigen in the subject.

45. A method of inducing an immune response against an autologous antigen associated with a disorder in a human subject, comprising: administering to the subject an immunogenic composition comprising a first, autologous polypeptide coupled to a second, heterologous polypeptide, such that an immune response is induced to an autologous antigen in the subject.

46. The method of claim 45, wherein the composition is administered to the subject more than once.

47. The method of claim 45, wherein the immune response is a T-cell dependent antibody response.

48. The method of claim 45, wherein the antibody response comprises the production of antibodies of the IgG isotype that bind to the autologous antigen.

49. The method of claim 45, wherein the autologous antigen is a cell-associated antigen.

50. The method of claim 45, wherein the autologous antigen is a soluble antigen.

51. The method of claim 45, wherein the autologous antigen is selected from the group consisting of: CD64, sL-selectin, elastase, sCD16, CD46, TNF-α, sTNF-R75, sTNF-R55, TGF-β, CD40, CD154, lipoprotein (a), CD56, IL-10, IFN-γ, IL-2, IL-2R, CD45, IL-4, IgE, EGFR, TGF-β, CD54, sCD44 v5, and CD95.

52. The method of claim 45, wherein the disorder is selected from the group consisting of: cancer, allergy, arthritis, atherosclerosis, graft rejection, and inflammatory disease.

53. The method of claim 46, wherein the autologous antigen is a tumor-associated antigen.

54. The method of claim 46, wherein the autologous antigen is expressed by a B cell.

55. A method of reducing the total amount or concentration of at least one class of antibody in the blood of a human subject comprising: administering to the human subject an immunogenic composition comprising a first, autologous polypeptide coupled to a second, heterologous polypeptide, wherein the first autologous polypeptide comprises at least a portion of a molecule expressed by a B cell of the human subject such that the total amount or concentration of at least one class of antibody in the blood of the human subject is reduced.

56. A method of reducing the number or concentration of cells expressing a cell-associated, autologous antigen in a subject, comprising: administering to the subject an immunogenic composition, comprising a first, autologous polypeptide coupled to a second, heterologous polypeptide, such that the number or concentration of cells expressing the cell-associated, autologous antigen are reduced.

57. The method of claim 56, wherein the number or concentration of cells in the subject is reduced by at least about 50% relative to the number or concentration of cells in an untreated subject.

58. The method of claim 57, wherein the cells are B cells.

59. A method of reducing the amount or concentration of a soluble autologous antigen present in a subject comprising: administering to the subject an immunogenic composition comprising a first, autologous polypeptide coupled to a second, heterologous polypeptide, such that an immune response is induced to a soluble autologous antigen in the subject.


SEQUENCE LISTING (2 sequences)

SEQ ID NO: 1 - protein, 338 aa, Homo sapiens
MAAEDELQLPRLPELFETGRQLLDEVEVATEPAGSRIVQEKVFKGLDLLEKAAEMLSQLDLFSRNEDLEEIASTDLKYLLVPAFQGALTMKQVNPSKRLDHLQRAREHFINYLTQCHCYHVAEFELPKTMNNSAENHTANSSMAYPSLVAMASQRQAKIQRYKQKKELEHRLSAMKSAVESGQADDERVREYYLLHLQRWIDISLEEIESIDQEIKILRERDSSREASTSSSRQERPPVKPFILTRNMAQAKVFGAGYPSLPTMTVSDWYEQHRKYGALPDQGIAKAAPEEFRKAAQQQEEQEEKEEEDDEQTLHRAREWDDWKDTHPRGYGNRQNMG

SEQ ID NO: 2 - protein, 133 aa, Mus musculus
VPAMTSSDLPLNFQGSPCSQIWQHPRFAAKKRSSMVKFHCYTNHSGALTWFRKRGSQQPQELVSEEGRIVQTQNGSVYTLTIQNIQYEDNGIYFCKQKCDSANHNVTDSCGTELLVLGFSTLDQLKRRNTLKD